SEQUENCE LISTING (14 sequences)

SEQ ID NO: 1 - protein, 250 aa, Human sapiens
  MISC_FEATURE: Human CD58 isoform 1
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 2 - protein, 237 aa, Hommo sapiens
  MISC_FEATURE: Human CD58 isoform 2
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNVL

SEQ ID NO: 3 - protein, 484 aa, Artificial sequence
  Synthetic construct
  MISC_FEATURE: CD22 svFv (M971)-41BB-Z
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 452 aa, Artificial sequence
  Synthetic construct
  MISC_FEATURE: CD19 scFv-28tm-CD2
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 5 - protein, 258 aa, Artificial sequence
  Synthetic construct
  MISC_FEATURE: m971 scFv plus leader of SEQ ID NO 3
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 6 - protein, 72 aa, Artificial sequence
  Synthetic construct
  MISC_FEATURE: Hinge/TM and restriction enzyme site of SEQ ID NO 3
AAATTTPAPRPPTPAPTIASQPLSLRPEACRPAAGGAVHTRGLDFACDIYIWAPLAGTCGVLLLSLVITLYC

SEQ ID NO: 7 - protein, 42 aa, Artificial sequence
  Synthetic construct
  MISC_FEATURE: costimulatory domain of SEQ ID NO 3
KRGRKKLLYIFKQPFMRPVQTTQEEDGCSCRFPEEEEGGCEL

SEQ ID NO: 8 - protein, 267 aa, Artificial sequence
  Synthetic construct
  MISC_FEATURE: CD19 scFv plus leader of SEQ ID NO 4
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 9 - protein, 69 aa, Artificial sequence
  Synthetic construct
  MISC_FEATURE: Hinge/TM and restriction enzyme site of SEQ ID NO 4
AAAIEVMYPPPYLDNEKSNGTIIHVKGKHLCPSPLFPGPSKPFWVLVVVGGVLACYSLLVTVAFIIFWV

SEQ ID NO: 10 - protein, 116 aa, Artificial sequence
  Synthetic construct
  MISC_FEATURE: costimulatory domain of SEQ ID NO 4
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 11 - protein, 26 aa, Artificial sequence
  Synthetic construct
  MISC_FEATURE: connector sequence
RKRREFATNFSLLKQAGDVEENPGPL

SEQ ID NO: 12 - DNA, 81 bp, Artificial sequence
  Synthetic construct
  MISC_FEATURE: encodes connector sequence of SEQ ID NO 11
cgcaagagaagagaattcgcaacaaacttctctctgctgaaacaagccggagatgtcgaagagaatcctggaccgctcgag

SEQ ID NO: 13 - protein, 4 aa, Artificial sequence
  Synthetic construct
  MISC_FEATURE: furin cleavage sequence
RKRR

SEQ ID NO: 14 - protein, 19 aa, Artificial sequence
  Synthetic construct
  MISC_FEATURE: auproteolytic peptide sequence P2A
ATNFSLLKQAGDVEENPGP

(19) United States
(12) Patent Application Publication
(10) Pub. No.: US 2025/0281532 A1
(43) Pub. Date: Sep. 11, 2025

(54) METHODS FOR DIAGNOSING OR TREATING HEALTH CONDITIONS OR OPTIMIZING THERAPEUTIC EFFICACY OF CAR-T CELLS THERAPIES

(71) Applicant: The Board of Trustees of the Leland Stanford Junior University, Stanford, CA (US)

(72) Inventors: Robbie MAJZNER, Palo Alto, CA (US); Crystal L. MACKALL, Stanford, CA (US); Christopher MOUNT, Stanford, CA (US); Michelle MONJE-DEISSEROTH, Stanford, CA (US); David B. MIKLOS, Stanford, CA (US); Matthew FRANK, Redwood City, CA (US); Yasodha NATKUNAM, Stanford, CA (US); Arash Ash ALIZADEH, Stanford, CA (US); Brian SWORDER, Stanford, CA (US); David M. KURTZ, Stanford, CA (US)

(21) Appl. No.: 18/251,697

(22) PCT Filed: Nov. 4, 2021
(86) PCT No.: PCT/US2021/058102

§ 371 Date: May 3, 2023

Related U.S. Application Data
(60) Provisional application No. 63/109,611, filed on Nov. 4, 2020.

(51) Int. Cl. A61K 35/17; A61K 40/11; A61K 40/31; A61K 40/42; A61P 35/00; C07K 14/705; C07K 16/28; C12Q 1/6827; C12Q 1/6886; G01N 33/68; G16H 10/60; G16H 15/00; G16H 20/17; G16H 50/30

(52) U.S. Cl. CPC A61K 35/17; A61K 40/11; A61K 40/31; A61K 40/4211; A61P 35/00; C07K 14/70507; C07K 16/2803; C12Q 1/6827; C12Q 1/6886; G01N 33/6872; G16H 10/60; G16H 15/00; G16H 20/17; G16H 50/30; C07K 2317/622; C12Q 2600/106; C12Q 2600/156; G01N 2333/70528; G01N 2800/52

(57) ABSTRACT

The present disclosure generally relates to, inter alia, methods, kits, and systems for the diagnosis and/or treating various health conditions, such as proliferative disorders (e.g., cancers), associated with a decreased level or a loss of CD58 expression or molecular alterations in CD58 activity.

### CROSS-REFERENCE TO RELATED APPLICATION

The present application is a U.S. National Phase Application of PCT International Application No. PCT/US2021/055185, filed on Oct. 15, 2021, designating the United States of America, which is an International Application of and claims the benefit of priority to U.S. Provisional Patent Application Ser. No. 63/109,611, filed on Nov. 4, 2020. The disclosures of the above-referenced applications are expressly incorporated by reference herein in their entirety, including any drawings.

### STATEMENT REGARDING FEDERALLY SPONSORED RESEARCH AND DEVELOPMENT

This invention was made with Government support under contracts CA241076 and CA049605 awarded by the National Institutes of Health. The Government has certain rights in the invention.

### INCORPORATION OF THE SEQUENCE LISTING

This application contains a Sequence Listing, which is hereby incorporated herein by reference in its entirety. The accompanying Sequence Listing text file, named “Sequence Listing_078430-525N01US_ST25.txt,” was created on May 2, 2023 and is 5 KB.

### FIELD

The present disclosure generally relates to, inter alia, methods, kits, and systems for the diagnosis and/or treating health conditions, such as proliferative disorders (e.g., cancers), associated with a decreased level or a loss of CD58 expression or molecular alterations in CD58 activity.

### BACKGROUND

Adoptive transfer of genetically modified immune cells has emerged as a potent therapy for various malignancies. For example, current modalities of adoptive T cell therapy include cells modified to express receptors specific for cancer antigens, such as chimeric antigen receptors (CARs) and high-affinity T cell receptors (TCRs). In adoptive T cell therapies, modified T cells can be activated by exposure to the cognate antigen in vitro or ex vivo, expanded, and then administered to the individual, where they proliferate and exhibit cytolytic activity and/or send signals to initiate an immune response against the cancer.

Recent developments using chimeric antigen receptor (CAR) modified autologous T cell (CART) therapy, which relies on redirecting T cells to a suitable cell-surface molecule on cancer cells such as B cell malignancies, have shown promising results in harnessing the power of the immune system to treat B cell malignancies and other cancers. For example, recent clinical trials using CAR-T cells specific for the CD19 molecule on B-cell malignancies demonstrated marked disease regression in a subset of patients with advanced cancers. A single dose of CD19 CAR-T cells results in complete remissions in approximately 50% of patients with large B-cell lymphoma (LBCL). This success has led to the FDA approval of two CD19-CAR T cell therapeutic agents, axicabtagene ciloleucel (YESCARTA®) and tisagenlecleucel (KYMRIAH®), with others in clinical development of medication for the treatment of LBCL and B-cell acute lymphoblastic leukemia (B-ALL). In particular, complete responses are sustained in a majority of LBCL patients.

However, besides the ability for the CAR-T cells to recognize and destroy the targeted cells, a successful therapeutic T cell therapy needs to have the ability to proliferate, to persist over time, and to further monitor for leukemic cell escapees. The variable phenotypic state of T cells, whether it is in a state of anergy, suppression, or exhaustion, have been reported to have various effects on CAR-T cells' efficacy. To be effective, CAR-T cells need to persist and maintain the ability to proliferate in response to the CAR's antigen.

In addition, an urgent therapeutic need exists to identify the cause of disease progression, and to treat patients who develop resistance to existing CAR-T therapies. In particular, it has been reported that CD19 loss appears to be the most common cause of relapse after CAR-T cell therapies for B-ALL, accounting for more than 90% of relapses in one series, and also occurs in up to 30% of cases of LBCL. This resistance has been observed only recently as post-therapy biopsies have become standard to determine patient specific factors driving therapeutic resistance.

Thus, new compositions and strategies are needed for generating improved therapeutic cells for adoptive T cell therapy. The presently disclosed aspects and embodiments address these needs and provide other related advantages.

### SUMMARY

Provided herein, inter alia, are methods, kits, and systems for the diagnosis and/or treatment of various health conditions such as proliferative disorders (e.g., cancers) associated with one or more molecular alterations in CD58 activity. In particular, some embodiments of the disclosure relate to methods for determining the responsiveness of an individual to a CAR-T cell therapy. Other embodiments relate to methods for identifying individuals who have an increased unresponsiveness to a CAR-T cell therapy. Also provided, in some embodiments, are methods for optimizing the therapeutic efficacy of a CAR-T cell therapy in individuals in need thereof. Additional embodiments of the disclosure relate to methods for administering a CAR-T cell therapy to individuals in need thereof. Also provided are kits and systems for the prevention and/or treatment of a health condition in individuals in need thereof.

In one aspect, provided herein are kits for the diagnosis and/or treatment of health conditions in an individual, the kit including (i) reagents for assessing an expression level of CD58 or the presence and/or absence of one or more molecular alterations of a CD58-encoding gene or a product thereof in a biological sample, and (ii) instructions for use thereof.

Non-limiting exemplary embodiments of the disclosed kits can include one or more of the following features. In some embodiments, the kit of the disclosure is further configured for determining the responsiveness of the individual to a CAR-T cell therapy, wherein the determining comprises: (a) detecting whether the expression level of CD58 is decreased or lost or one or more molecular alterations in the CD58-encoding gene or a product thereof is present in a biological sample obtained from the individual, wherein said detecting comprises contacting the biological sample with a detection reagent and detecting an interaction between the detection reagent with a CD58 encoding gene or a product thereof in the sample; and (b) identifying the individual as having decreased responsiveness to treatment with the CAR-T cell therapy if the expression level of CD58 is decreased compared to a reference expression level of CD58 or lost or at least one of the one or more molecular alterations in CD58 activity is detected in the sample.

In some embodiments, the kit of the disclosure is further configured for identifying an individual who has an increased unresponsiveness to a CAR-T cell therapy, wherein the identifying comprises: (a) detecting whether the expression level of CD58 is decreased or lost or one or more molecular alterations in the CD58-encoding gene or a product thereof is present in a biological sample obtained from the individual, wherein said detecting comprises contacting the biological sample with a detection reagent and detecting an interaction between the detection reagent with the CD58 encoding gene or a product thereof in the sample; and (b) selecting the individual as having increased unresponsiveness to treatment with the CAR-T cell therapy if the expression level of CD58 is decreased compared to a reference expression level of CD58 or lost or at least one of the one or more molecular alterations in CD58 activity is detected in the sample; or selecting the individual as not having increased unresponsiveness to treatment with the CAR-T cell therapy if the expression level of CD58 is not decreased compared to a reference expression level of CD58 nor lost or none of the one or more molecular alterations in CD58 activity is detected in the sample.

In some embodiments, the kit of the disclosure is further configured for optimizing the therapeutic efficacy of a CAR-T cell therapy in an individual, wherein the optimizing comprises: (a) detecting whether the expression level of CD58 is decreased compared to a reference expression level of CD58 or lost or one or more molecular alterations in the CD58-encoding gene or a product thereof is present in a biological sample obtained from the individual, wherein said detecting comprises contacting the biological sample with a detection reagent and detecting an interaction between the detection reagent with a CD58 encoding gene or a product thereof in the sample; and (b) identifying a therapeutically effective amount of the CAR-T cell therapy based on the detected interaction between the detection reagent with the CD58 encoding gene or a product thereof.

In some embodiments, the individual has or is suspected of having a health condition associated with a decreased level or loss of CD58 expression compared to the reference expression level of CD58 or with one or more molecular alterations in the CD58-encoding gene or a product thereof. In some embodiments, the health condition is a proliferative disorder selected from the group consisting of a solid tumor cancer, a non-solid tumor cancer, and a hematological malignancy. In some embodiments, the health condition is a cancer, optionally non-Hodgkin's lymphoma, Burkitt's lymphoma, small lymphocytic lymphoma, large B-cell lymphoma (LBCL), primary effusion lymphoma, diffuse large B-cell lymphoma, splenic marginal zone lymphoma, MALT (mucosa-associated lymphoid tissue) lymphoma, hairy cell leukemia, chronic lymphocytic leukemia, B-cell prolymphocytic leukemia, B cell lymphomas, Hodgkin's disease, B cell non-Hodgkin's lymphoma (NHL), leukemias, acutelymphoblastic leukemia (ALL), chronic lymphocytic leukemia (CLL), B-cell chronic lymphocytic leukemia (B-CLL), hairy cell leukemia, chronic myoblastic leukemia, or myeloma.

In some embodiments, the one or more molecular alterations in the CD58-encoding gene or a product thereof is selected from the group consisting of an increased RNA/protein expression, a reduced RNA/protein expression, a loss of expression, an aberrant RNA/protein expression, a single nucleotide point mutation (SNP), a single-nucleotide variation (SNV), a gene amplification, a gene rearrangement, a gene fusion, a deletion, a frameshift deletion, an insertion, an InDel mutation, an epigenetic alteration, an amino acid substitution, and combinations of any thereof. In some embodiments, the one or more molecular alterations comprises a loss of CD58 expression, a reduced expression of CD58 compared to the reference expression level of CD58, or expression of a mutated form of CD58. In some embodiments, the one or more molecular alterations comprises an amino acid substitution at a position corresponding to K60 of SEQ ID NO: 1. In some embodiments, the amino acid substitution is a Lys-to-Glu substitution (K60E). In some embodiments, the one or more molecular alterations comprises an amino acid substitution at a position corresponding to C187 of SEQ ID NO: 1. In some embodiments, the amino acid substitution is a Cys-to-Arg substitution (C187R). In some embodiments, the one or more molecular alterations in the CD58-encoding gene or a product thereof includes a reduced binding affinity of the CD58 protein product to its ligand CD2.

In some embodiments, the assessing comprises using a nucleic-acid-based analytical assay selected from the group consisting of cancer personalized profiling by deep sequencing (CAPP-seq), nucleic acid sequencing, circulating tumor nucleic acid assessment, next generation sequencing (NGS), nucleic acid amplification-based assays, loop-mediated isothermal amplification (LAMP), rolling circle amplification (RCA), polymerase chain reaction (PCR), real-time PCR, quantitative reverse transcription PCR (qRT-PCR), PCR-RFLP assay, HPLC, mass-spectrometric genotyping, nucleic acid hybridization assay, comparative genomic hybridization, fluorescent in-situ hybridization (FISH), restriction digestion, capillary electrophoresis, and combinations of any thereof. In some embodiments, the assessing comprises using a protein-based analytical assay selected from the group consisting of immunohistochemistry (IHC), protein-microarray, western blotting, mass spectrometry, flow cytometry, enzyme-linked immunosorbent assay (ELISA), immunofluorescence staining, multiplex detection assay, and combinations of any thereof.

In some embodiments, the kit of the disclosure is further configured for treatment of the health condition. In some embodiments, the CAR-T cell therapy is administered to individual as single therapy or in combination with one or more additional therapies. In some embodiments, the CAR-T cell therapy and/or at least one additional therapy comprises a CAR construct comprising a CD2 signaling domain. In some embodiments, the CAR-T cell therapy targets an antigen that is expressed at low density compared to a density in a wild-type cell.

In another aspect, provided herein are genetic-based systems for diagnosis and treatment of health conditions, the systems include: a) a logic processor; and b) a stored program code that is executable by the logic processor, which when executed by the processor provides operations for performing a method according to present disclosure. In some embodiments, the systems include (a) a logic processor; and (b) a stored program code that is executable by the logic processor, which when executed by the processor provides operations for performing one or more of the following: (i) determining the responsiveness of an individual to a CAR-T cell therapy; (ii) identifying an individual as having increased unresponsiveness to treatment with a CAR-T cell therapy; (iii) optimizing the therapeutic efficacy of a CAR-T cell therapy in an individual; and (iv) calculating or administering a therapeutically effective amount of a CAR-T cell therapy to an individual.

In some embodiments, the systems disclosed herein further include a report engine communicatively coupled to the logic processor, wherein reports produced by the report engine depend upon results from execution of the program code, wherein the program code configures the logic processor to receive from the genetic scanner a preselected set of data input pertaining to an expression level of CD58 or the presence and/or absence of one or more molecular alterations of a CD58-encoding gene or a product thereof in a biological obtained from an individual in order to assign a relative performance score to the individual's responsiveness to the CAR-T cell therapy based at least in part on the preselected set of data input, and optionally: (a) determining the responsiveness of the individual to the CAR-T cell therapy; (b) identifying the individual as having increased unresponsiveness to treatment with the CAR-T cell therapy; (c) optimizing the therapeutic efficacy of the CAR-T cell therapy in the individual; and/or (d) calculating or administering a therapeutically effective amount of the CAR-T cell therapy to the individual.

In some embodiments, the systems of the disclosure further include generating a report that contains information relevant to the individuals identified as having increased unresponsiveness to the CAR-T cell therapy and/or relevant to the CAR-T cell therapy identified as being effective for treatment of health conditions. In some embodiments, the profile report is characterized as having an encoding selected from the group consisting of “.doc”; “.pdf”; “.xml”; “.html”; “.jpg”; “.aspx”; “.php”, and a combination of any thereof.

In yet another aspect, provided herein is a non-transitory computer readable medium containing machine executable instructions that when executed cause a processor to perform operations including: receiving a report including a preselected set of data input; assigning, based at least in part on the report, a relative performance score to the identified CAR-T cell therapy; and outputting a report for the CAR-T cell therapy based upon the assigned performance score. In some embodiments, provided herein is a non-transitory computer readable medium containing machine executable instructions that when executed cause a processor to perform operations including: receiving a report including a preselected set of data input; assigning, based at least in part on the report, a relative unresponsiveness score to the identified individual; and outputting a report for the individual based upon the assigned unresponsiveness score. Accordingly, CAR-T cell therapy reports and individual reports generated by the systems of the disclosure are also with the scope of this disclosure.

In one aspect, provided herein are methods for determining the responsiveness of an individual to a CAR-T cell therapy, the method including: (a) detecting whether an expression level of CD58 is decreased compared to a reference expression level of CD58 or lost or one or more molecular alterations in CD58 activity is present in a biological sample obtained from the individual, wherein said detecting includes contacting the biological sample with a detection reagent and detecting an interaction between the detection reagent with a CD58 encoding gene or a product thereof in the sample; and (b) identifying the individual as having decreased responsiveness to treatment with the CAR-T cell therapy if an expression level of CD58 is decreased compared to a reference expression level of CD58 or lost or at least one of the one or more molecular alterations in CD58 activity is detected in the sample.

In another aspect, provided herein are methods for identifying an individual who has an increased unresponsiveness to a CAR-T cell therapy, the method including: (a) detecting whether an expression level of CD58 is decreased compared to a reference expression level of CD58 or lost or one or more molecular alterations in CD58 activity is present in a biological sample obtained from the individual, wherein said detecting includes contacting the biological sample with a detection reagent and detecting an interaction between the detection reagent with the CD58 encoding gene or a product thereof in the sample; and (b) selecting the individual as having increased unresponsiveness to treatment with a CAR-T cell therapy if an expression level of CD58 is decreased or lost or at least one of the one or more molecular alterations in CD58 activity is detected in the sample; or selecting the individual as not having increased unresponsiveness to treatment with the CAR-T cell therapy if the expression level of CD58 is not decreased nor lost or none of the one or more molecular alterations in CD58 activity is detected in the sample.

In another aspect, provided herein are methods for optimizing the therapeutic efficacy of a CAR-T cell therapy in an individual, the method including: (a) detecting whether an expression level of CD58 is decreased compared to a reference expression level of CD58 or lost or one or more molecular alterations in CD58 activity is present in a biological sample obtained from the individual, wherein said detecting includes contacting the biological sample with a detection reagent and detecting an interaction between the detection reagent with a CD58 encoding gene or a product thereof in the sample; (b) identifying a therapeutically effective amount of the CAR-T cell therapy based on the detected interaction between the detection reagent with the CD58 encoding gene or a product thereof.

In yet another aspect, provided herein are methods for administering a CAR-T cell therapy to an individual, the method including: (a) detecting whether an expression level of CD58 is decreased compared to a reference expression level of CD58 or lost or one or more molecular alterations in CD58 activity is present in a biological sample obtained from the individual, wherein said detecting includes contacting the biological sample with a detection reagent and detecting an interaction between the detection reagent with a CD58 encoding gene or a product thereof in the sample; (b) administering a therapeutically effective amount of the CAR-T cell therapy based on the detected interaction between the detection reagent with the CD58 encoding gene or a product thereof.

Non-limiting exemplary embodiments of the disclosed methods can include one or more of the following features. In some embodiments, the individual has or is suspected of having a health condition associated with a decreased level or loss of CD58 expression or with one or more molecular alterations in CD58 activity. In some embodiments, the methods of the disclosure further include treating the health condition. In some embodiments, the health condition is a proliferative disorder selected from the group consisting of a solid tumor cancer, a non-solid tumor cancer, and a hematological malignancy. In some embodiments, the health condition is a cancer, optionally non-Hodgkin's lymphoma, Burkitt's lymphoma, small lymphocytic lymphoma, large B-cell lymphoma (LBCL), primary effusion lymphoma, diffuse large B-cell lymphoma, splenic marginal zone lymphoma, MALT (mucosa-associated lymphoid tissue) lymphoma, hairy cell leukemia, chronic lymphocytic leukemia, B-cell prolymphocytic leukemia, B cell lymphomas, Hodgkin's disease, B cell non-Hodgkin's lymphoma (NHL), leukemias, acutelymphoblastic leukemia (ALL), chronic lymphocytic leukemia (CLL), B cell chronic lymphocytic leukemia (BCLL), hairy cell leukemia, chronic myoblastic leukemia, or myeloma.

In some embodiments, the one or more molecular alterations in CD58 activity is selected from the group consisting of an increased RNA/protein expression, a reduced RNA/protein expression, a loss of expression, an aberrant RNA/protein expression, a single nucleotide point mutation (SNP), a single-nucleotide variation (SNV), a gene amplification, a gene rearrangement, a gene fusion, a deletion, a frameshift deletion, an insertion, an InDel mutation, an epigenetic alteration, an amino acid substitution, and combinations of any thereof. In some embodiments, the one or more molecular alterations includes a loss of CD58 expression, a reduced expression of CD58 compared to a reference expression level of CD58, or expression of a mutated form of CD58. In some embodiments, the one or more molecular alterations includes an amino acid substitution at a position corresponding to K60 of SEQ ID NO: 1. In some embodiments, the amino acid substitution is a Lys-to-Glu substitution (K60E). In some embodiments, the one or more molecular alterations includes an amino acid substitution at a position corresponding to C187 of SEQ ID NO: 1. In some embodiments, the amino acid substitution is a Cys-to-Arg substitution (C187R). In some embodiments, the one or more molecular alterations in the CD58-encoding gene or a product thereof comprises a reduced binding affinity of the CD58 protein product to its ligand CD2.

In some embodiments, the detection of the interaction between the detection reagent with the CD58 encoding gene or a product thereof includes using a nucleic-acid-based analytical assay selected from the group consisting of cancer personalized profiling by deep sequencing (CAPP-seq), nucleic acid sequencing, circulating tumor nucleic acid assessment, next generation sequencing (NGS), nucleic acid amplification-based assays, loop-mediated isothermal amplification (LAMP), rolling circle amplification (RCA), polymerase chain reaction (PCR), real-time PCR, quantitative reverse transcription PCR (qRT-PCR), PCR-RFLP assay, HPLC, mass-spectrometric genotyping, nucleic acid hybridization assay, comparative genomic hybridization, fluorescent in-situ hybridization (FISH), restriction digestion, capillary electrophoresis, and combinations of any thereof.

In some embodiments, the detection of the interaction between the detection reagent with the CD58 encoding gene or a product thereof includes using a protein-based analytical assay selected from the group consisting of immunohistochemistry (IHC), protein-microarray, western blotting, mass spectrometry, flow cytometry, enzyme-linked immunosorbent assay (ELISA), immunofluorescence staining, multiplex detection assay, and combinations of any thereof.

In some embodiments, the methods described herein further include administering the CAR-T cell therapy to the individual, wherein the CAR-T cell therapy is administered to individual as single therapy or in combination with one or more additional therapies. In some embodiments, the CAR-T cell therapy and/or at least one additional therapy includes a CAR construct including a CD2 costimulatory domain. In some embodiments, the CAR-T cell therapy targets an antigen that is expressed at low density compared to a density in a wild-type cell.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1E graphically summarize the results of experiments performed to demonstrate that durable remission of in LBCL patients receiving axicabtagene ciloleucel requires no alterations in CD58. Patients with mutations in CD58 or loss of expression of CD58 by immunohistochemistry (IHC) have poor outcomes. CR: complete response; PR: partial response; SD: stable response; PD: progressive disease.

FIGS. 2A-2J graphically summarize the results of experiments performed to illustrate that a loss in CD58 expression diminishes the efficacy of CAR-T cells in vitro and in xenograft models.

FIGS. 3A-3J graphically summarize the results of experiments performed to illustrate that CD58-CD2 interactions are responsible for enhanced CAR-T cell activity.

FIGS. 4A-4J graphically summarize the results of experiments performed to illustrate that CAR-T cells can be engineered to overcome CD58 loss in B cell malignancies.

FIGS. 5A-5B graphically summarize the results of experiments performed to illustrate that patients with either loss of expression by immunohistochemistry (IHC) (FIG. 5A) or with mutations in CD58 (FIG. 5B) have poor outcomes after CAR-T cell therapy.

FIGS. 6A-6B graphically summarize the results of experiments performed to illustrate the reduction in cytokine production by a GD2-4-1BBζ CAR when incubated with DIPG cell lines with and without CD58 knockout.

FIGS. 7A-7C graphically summarize the results of experiments performed to evaluate in vitro CAR efficacy of CD19-CD28ζ or CD19-4-1BBζ against CD58-wildtype or CD58-knockout lines, as well as CAR efficacy in vivo against CD58-knockout xenografts and wild-type xenografts.

FIGS. 8A-8B graphically summarize the results of experiments performed to illustrate that proteins involved in actin cytoskeleton reorganization and such as VASP and WAS were elevated in CD2 stimulated cells.

### DETAILED DESCRIPTION OF THE DISCLOSURE

The present disclosure generally relates to, inter alia, methods, kits, and systems for the diagnosis and/or treatment of various health conditions such as proliferative disorders (e.g., cancers) associated with one or more molecular alterations in CD58 activity. In particular, some embodiments of the disclosure relate to methods for determining the responsiveness of an individual to a CAR-T cell therapy. Some embodiments of the disclosure relate to methods for identifying individuals who have an increased unresponsiveness to a CAR-T cell therapy. Other embodiments of the disclosure concern methods for optimizing the therapeutic efficacy of a CAR-T cell therapy in individuals in need thereof. Additional embodiments of the disclosure relate to methods for administering a CAR-T cell therapy to individuals in need thereof. Also provided are kits and systems for the prevention and/or treatment of a health condition in individuals in need thereof.

As discussed above, recent developments using CAR modified autologous T cell (CART) therapy, which relies on redirecting T cells to a suitable cell-surface molecule on cancer cells such as B cell malignancies, show promising results in harnessing the power of the immune system to treat B cell malignancies and other cancers. In particular, CD19 CAR-T cells have revolutionized the treatment of B cell malignancies including large B cell lymphoma (LBCL). For example, a single dose of CD19 CAR-T cells results in complete remissions in approximately 50% of patients with LBCL. This success has led to the FDA approval of two agents (axicabtagene ciloleucel and tisagenlecleucel), with others in clinical development. In particular, complete responses are sustained in a majority of LBCL patients.

Phase-two trials have demonstrated a durable complete response (CR) rate for CD19 CAR in large B-cell lymphoma (LBCL) of up to 40%, which is a significant improvement over prior standards of care. Based on these results and the real-world study of axicabtagene ciloleucel (YESTARTA®), a CD19-CAR-T cell therapy that has now become the standard of care for LBCL after failing two lines of chemotherapy. Ongoing clinical trials will determine if CD19 CAR T therapy will become the new standard for refractory or early-relapsed LBCL (NCT03391466, NCT03575351, and NCT03570892).

However, an urgent therapeutic need exists to identify the cause of disease progression, and to treat patients who develop resistance to existing CAR-T therapies. In particular, CD19 loss appears to be the most common cause of relapse after CAR-T cell therapies for B-cell acute lymphoblastic leukemia (B-ALL), accounting for more than 90% of relapses in one series, and also occurs in up to 30% of cases of LBCL. This resistance has been observed only recently as post-therapy biopsies have become standard to determine patient specific factors driving therapeutic resistance. A thorough understanding of the mechanisms of CAR resistance will be helpful in determining which patients are most likely to benefit from CAR-T cells and in order to generate novel constructs that are able to extend benefit to a larger number of patients. Without being bound to any particular theory, because a high percentage of complete responses to CD19 CAR in LBCL are durable, it is contemplated that increases in the CR rate will translate to curing more patients of their disease.

Mechanisms of CAR-T cell efficacy are poorly understood. It is generally thought that the integration of costimulatory domains into second generation constructs has resulted in the clinical success observed with CD19 CAR. However, recent study has shown that some of the perceived benefits of second generation CARs in preclinical models may have actually been driven by other elements of the CAR architecture. As described in greater detail below, the experimental results presented herein demonstrate that even though they contain highly functional costimulatory domains, the FDA-approved CD19-CAR-T cell therapeutic agents axicabtagene ciloleucel and tisagenlecleucel both ultimately fail when T cells are deprived of additional costimulation by CD2 through CD58, its native ligand on tumor cells. However, the experimental data described herein also indicate that the detrimental effects of CD58 mutation or loss may be context dependent on the target antigen density expressed by tumor cells.

CD2 is an important costimulatory domain for the native TCR and its interaction with CD58 has previously been shown to support cytokine production after TCR ligation and also by first generation CARs in vitro. As described in greater detail below, several experiments described herein were performed to explore the role of CD2 ligation in the context of CAR-T cells and found that much like with the native TCR, CD2 ligation by CD58 results in enhanced phosphorylation of proximal TCR molecules, but also changes cytoskeletal and adhesion molecules important for T cell activity. The data presented herein aligns with recently published work on the phosphopeptidome of cytotoxic T lymphocytes activated through CD2 in which CD2 was determined to drive cytoskeletal rearrangements resulting in tumor cell lysis. Additionally, phosphorylation of the CARD 11-BCL10-MALT1 (CBM) signalosome provides a potential mechanism to explain sustained anti-tumor activity towards target cells expressing CD58.

Volumes of research have focused on generating more functional CARs by integrating additional costimulatory and cytokine signals into CAR constructs. However, this work has largely been guided by preclinical modeling that may not capture true mechanisms of resistance in patients. It may be essential that as CARs are deployed for more patients with additional indications including solid tumors, deep correlative work is carried out that can lead researchers to understand mechanisms of CAR failure. After observing limited activity of CD19 CARs in patients with CD58 aberrations and in preclinical models in which CD58 was knocked out, additional experiments were performed to generate CAR-T cells that are capable of overcoming this novel resistance mechanism through integration of CD2 signaling. It was found that CAR-T cells in which CD2 costimulation is provided in trans to the traditional CAR were successful in overcoming CD58 loss in vivo. The experimental result presented herein also indicates that costimulation may best be provided to CAR constructs in trans, as occurs in the natural TCR setting and has been previously reported for CARs with alternative costimulation domains.

CD58 mutations and changes in expression are common in other cancers, including Hodgkin's lymphoma, chronic lymphocytic leukemia, multiple myeloma, and even colon cancer. Thus, it is likely that this axis will be an important determinant in CAR-T cell outcomes for other diseases and could also predict responses to other immunotherapy modalities including checkpoint inhibitors, bispecific antibodies, and transgenic TCRs. Additionally, CD2 ligation is also is important for natural killer (NK) cell function, indicating that CD58 mutations could limit the efficacy of CAR-NK cells, which have been recently credentialed as a promising modality for treatment of B cell malignancies. It is also probable that there are additional co-receptors on tumor cells that can also modulate CAR function and, like CD58, will emerge as important factors in predicting response to CAR-T cell therapy.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value±up to 10%, up to ±5%, or up to ±1%.

The terms “administration” and “administering”, as used herein, refer to the delivery of a bioactive composition or formulation by an administration route comprising, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

“Cancer” refers to the presence of cells possessing several characteristics typical of cancer-causing cells, such as uncontrolled proliferation, immortality, metastatic potential, rapid growth and proliferation rate, and certain characteristic morphological features. Cancer cells can aggregate into a mass, such as a tumor, or can exist alone within an individual. A tumor can be a solid tumor, a soft tissue tumor, or a metastatic lesion. As used herein, the term “cancer” also encompasses other types of non-tumor cancers. Non-limiting examples include blood cancers or hematological cancers, such as leukemia. Cancer can include premalignant, as well as malignant cancers.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “engineered” or “recombinant” nucleic acid molecule, polypeptide, or cell as used herein, refers to a nucleic acid molecule, polypeptide, or cell that has been altered through human intervention.

As used herein, and unless otherwise specified, a “therapeutically effective amount” or a “therapeutically effective number” of an agent is an amount or number sufficient to provide a therapeutic benefit in the treatment or management of a disease, e.g., cancer, or to delay or minimize one or more symptoms associated with the disease. A therapeutically effective amount or number of a compound means an amount or number of therapeutic agent, alone or in combination with other therapeutic agents, which provides a therapeutic benefit in the treatment or management of the disease. The term “therapeutically effective amount” can encompass an amount or number that improves overall therapy of the disease, reduces or avoids symptoms or causes of the disease, or enhances therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human subject) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

It is understood that aspects and embodiments of the disclosure described herein include “comprising,” “consisting,” and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### CD58

CD58, also known as the lymphocyte function-associated antigen-3 (LFA-3), was first identified from humans (Homo sapiens) as an adhesion molecule in the 1980s. It is a heavily glycosylated protein whose extracellular region contains a single V-set and a C2-set Ig superfamily (IgSF) domain. CD58 was expressed on the surface of human hemopoietic and non-hemopoietic lineages, including dendritic cells, macrophages, endothelial cells, and erythrocytes in a transmembrane and glycosylphosphatidylinositol (GPI)-anchored form. CD58 was also identified from several other mammals, including porcine (Sus scrofa) and sheep (Ovis aries). Several previous studies in humans have shown the involvement of CD58 in T-cell cytokine production, T-cell responsiveness to IL-12, induction of TNF-α and IL-10 from monocytes, and IgE production by B cells. Blockade of CD58 by anti-CD58 monoclonal antibodies and a CD58-Ig fusion protein can reduce inflammatory responses and diminish the recognition and cytolysis of target cells by cytotoxic T lymphocytes and NK cells. These findings suggest that CD58 plays important roles in both innate and adaptive immunities, with a particularly regulatory role at the effector and target cell levels.

CD58 and CD2 have been known as a pair of reciprocal adhesion molecules involved in the immune modulations in a variety of cell types, such as CD8+ T cell and NK cells. In particular, the CD2 pathway can directly mediate CD3− independent T cell activation and has a costimulatory role in a variety of immune cell types, such as CD8+ T cell and NK cells, and therefore this pair of adhesion molecules is involved in the immune modulation of CD8+ T cell and NK cell-mediated cellular immunity in humans and several other mammals. In most cases, CD58 exerts its functions through the interaction with its receptor CD2, which has also been known as lymphocyte function-associated antigen-2 (LFA-2). CD2 is also a member of the immunoglobulin superfamily, which is expressed on the surface of almost all mature peripheral T cells, thymocytes, NK cells, and thymic B cells. The interaction of CD58 with CD2 may be found to be essential for the activation of cellular immunity, such as CD8+ cytotoxic T lymphocytes and NK cell-mediated cytotoxic reactions.

In human, CD58 has multiple isoforms ranging in size from 55,000 to 75,000 da, depending on alternative splicing and sugar chain additions. CD58 consists of two extracellular domains and one transmembrane domain and is expressed in almost all cells, especially on the surface of antigen presenting cells, in particular macrophages and hematopoietic cells including B-cells. CD58 expression is increased by cytokine stimulation. It mediates cellular adhesion and participates in signal transduction when it binds to its ligand, the CD2 (LFA-2). Cellular interactions regulated by the CD58/CD2 axis are involved in the antigen-independent adhesion pathway and cytotoxic T lymphocyte (CTL) activity. CD58 has two isoforms. One isoform is anchored in the cell membrane by a glycophosphatidyl inositol tail, while the other has a transmembrane hydrophobic segment and a cytoplasmic segment composed of 12 amino acids. In addition, only the first domain of the two extracellular domains binds to CD2 on the surface of the T lymphocytes, placing the T lymphocytes and antigen presenting cells close together, enabling the T lymphocytes to generate an immune response. The amino acid sequence of CD58 isoform 1 (also known as long isoform) is shown below.

Human CD58 isoform 1 (SEQ ID NO: 1):
MVAGSDAGRA LGVLSVVCLL HCFGFISCFS QQIYGVVYGN
VTFHVPSNVP LKEVLWKKQK DKVAELENSE FRAFSSFKNR
VYLDTVSGSL TIYNLTSSDE DEYEMESPNI TDTMKFFLYV
LESLPSPTLT CALINGSIEV QCMIPEHYNS

HRGLIMYSWD CPMEQCKRNS TSIYFKMEND LPQKIQCTLS NPLFNTTSSI ILTTCIPSSG HSRHRYALIP IPLAVITTCI VLYMNGILKC DRKPDRTNSN

The amino acid sequence of human CD58 isoform 2 (also known as short isoform; SEQ ID NO: 2) is similar to SEQ ID NO: 1 above, with the exception that amino acid residues 236-237 are VL (instead of GI), and that amino acid residues 238-250 are missing. This CD58 isoform 2 is a translation product of a transcript variant which includes an alternate segment in the 3′ coding region compared to variant 1 (which encodes the isoform 1) that causes a frameshift. The resulting protein (isoform 2) has a distinct C-terminus, compared to isoform 1. This transcript variant (2) also contains a unique 3′ UTR compared to transcript variant 1.

Human CD58 isoform 2 (SEQ ID NO: 2):
MVAGSDAGRA LGVLSVVCLL HCFGFISCFS QQIYGVVYGN
VTFHVPSNVP LKEVLWKKQK DKVAELENSE FRAFSSFKNR
VYLDTVSGSL TIYNLTSSDE DEYEMESPNI TDTMKFFLYV
LESLPSPILT CALINGSIEV QCMIPEHYNS HRGLIMYSWD
CPMEQCKRNS TSIYFKMEND LPQKIQCTLS NPLENTTSSI
ILTTCIPSSG HSRHRYALIP IPLAVITTCI VLYMNVL

CD58 is known to be involved in cytotoxic activity expression or antigen presentation reaction by binding to CD2 (LFA-2). In particular, CD58's binding to CD2, e.g., on T cells, is important in strengthening the adhesion between the T cells and professional antigen presenting cells (APCs). This adhesion occurs as part of the transitory initial encounters between T cells and APCs before T cell activation, when T cells are roaming the lymph nodes looking at the surface of APCs for peptide:MHC complexes the T-cell receptors are reactive to.

Mutations of CD58 have been linked to immune evasion observed in some lymphomas and studies are underway to analyze how its involvement directly affects classical Hodgkin lymphoma. Polymorphisms in the CD58 gene are associated with increased risk for multiple sclerosis. For example, genomic region containing the single-nucleotide polymorphism rs1335532, associated with high risk of multiple sclerosis, has enhancer properties and can significantly boost the CD58 promoter activity in lymphoblast cells. The protective (C) rs1335532 allele creates functional binding site for ASCL2 transcription factor, a target of the Wnt signaling pathway. In addition, CD58 plays a role in the regulation of colorectal tumor-initiating cells. Thus, cells that express CD58 have become of interest in tumorigenesis.

While CD58 is highly expressed in B-ALL and serves as an important marker of minimal residual disease by flow cytometry in that disease, it is frequently mutated, downregulated, deleted, or silenced in LBCL. Additionally, CD58 loss has been previously linked to immune evasion in relapsed LBCL.

The role of CD58 expression on LBCL in determining response to CD19 CAR-T cell therapy is described in greater detail below. In particular, experiments have been designed and carried out to demonstrate that patients with loss of CD58 expression or with CD58 mutations do not achieve sustained responses to axicabtagene ciloleucel. In addition, using in vitro systems and murine xenografts, the functional importance of CD58 ligation has been investigated by generating CAR constructs and ultimately CAR-T cells capable of overcoming CD58 loss in LBCL. By dissecting resistance mechanisms in LBCL, the experimental results described herein illustrate the generation of novel therapeutics that are capable of extending durable remissions to a greater number of patients.

CD58, also known as the lymphocyte function-associated antigen-3 (LFA-3), mediates cellular adhesion and participates in signal transduction when it binds to its ligand, the CD2. Cellular interactions regulated by the CD58/CD2 antigens are involved in the antigen-independent adhesion pathway and cytotoxic T lymphocyte (CTL) activity. CD58 has two isoforms. One isoform is anchored in the cell membrane by a glycophosphatidyl inositol tail, while the other has a transmembrane hydrophobic segment and a cytoplasmic segment composed of 12 amino acids.

### Methods of the Disclosure

As described in greater detail herein, some embodiments of the present disclosure provide various methods for determining the responsiveness of an individual to a CAR-T cell therapy, the method including: (a) detecting whether an expression level of CD58 is decreased compared to a reference expression level of CD58 or lost or one or more molecular alterations in CD58 activity is present in a biological sample obtained from the individual, wherein said detecting includes contacting the biological sample with a detection reagent and detecting an interaction between the detection reagent with a CD58 encoding gene or a product thereof in the sample; and (b) identifying the individual as having decreased responsiveness to treatment with the CAR-T cell therapy if an expression level of CD58 is decreased or lost or at least one of the one or more molecular alterations in CD58 activity is detected in the sample.

In another aspect, provided herein are methods for identifying an individual who has an increased unresponsiveness to a CAR-T cell therapy, the method including: (a) detecting whether an expression level of CD58 is decreased compared to a reference expression level of CD58 or lost or one or more molecular alterations in CD58 activity is present in a biological sample obtained from the individual, wherein said detecting includes contacting the biological sample with a detection reagent and detecting an interaction between the detection reagent with the CD58 encoding gene or a product thereof in the sample; and (b) selecting the individual as having increased unresponsiveness to treatment with a CAR-T cell therapy if an expression level of CD58 is decreased or lost or at least one of the one or more molecular alterations in CD58 activity is detected in the sample; or selecting the individual as not having increased unresponsiveness to treatment with the CAR-T cell therapy if none of the one or more molecular alterations in CD58 activity is detected in the sample.

In another aspect, provided herein are methods for optimizing the therapeutic efficacy of a CAR-T cell therapy in an individual, the method including: (a) detecting whether an expression level of CD58 is decreased compared to a reference expression level of CD58 or lost or one or more molecular alterations in CD58 activity is present in a biological sample obtained from the individual, wherein said detecting includes contacting the biological sample with a detection reagent and detecting an interaction between the detection reagent with a CD58 encoding gene or a product thereof in the sample; (b) identifying a therapeutically effective amount of the CAR-T cell therapy based on the detected interaction between the detection reagent with the CD58 encoding gene or a product thereof.

In yet another aspect, provided herein are methods for administering a CAR-T cell therapy to an individual, the method including: (a) detecting whether an expression level of CD58 is decreased compared to a reference expression level of CD58 or lost or one or more molecular alterations in CD58 activity is present in a biological sample obtained from the individual, wherein said detecting includes contacting the biological sample with a detection reagent and detecting an interaction between the detection reagent with a CD58 encoding gene or a product thereof in the sample; (b) administering a therapeutically effective amount of the CAR-T cell therapy based on the detected interaction between the detection reagent with the CD58 encoding gene or a product thereof.

One aspect provides a method of treating an individual with a health condition characterized by at least one of: a decreased or lost expression of CD58 or one or more molecular alterations in the CD58-encoding gene, the method comprising: detecting whether expression level of CD58 is decreased compared to a reference expression level of CD58 or lost or one or more molecular alterations in the CD58-encoding gene or a product thereof is present in a biological sample obtained from the individual, wherein said detecting comprises contacting the biological sample with a detection reagent and detecting an interaction between the detection reagent with a CD58 encoding gene or a product thereof in the sample; identifying the individual as likely to respond to a treatment with a CAR construct comprising a CD2 signaling domain if the expression level of CD58 is decreased or lost compared to a reference expression level of CD58 or at least one of the one or more molecular alterations in CD58 activity is detected in the sample, and administering the treatment with a CAR construct comprising a CD2 signaling domain to the individual identified in step (b) as likely to respond to treatment with a CAR construct comprising a CD2.

In yet another aspect is provided a method of treating a health condition in an individual, the method comprising: detecting whether expression level of CD58 is decreased or lost compared to a reference expression level of CD58 or one or more molecular alterations in the CD58-encoding gene or a product thereof is present in a biological sample obtained from the individual, wherein said detecting comprises contacting the biological sample with a detection reagent and detecting an interaction between the detection reagent with a CD58 encoding gene or a product thereof in the sample; and administering a treatment with a CAR construct comprising a CD2 signaling domain to the individual based on the detection of a decreased or lost expression level of CD58 or one or more molecular alterations in the CD58-encoding gene in step (a).

In some embodiments, the reference expression level of CD58 can comprise the median level of expression of CD58 in samples from a group/population of patients. In some embodiments, the group/population of patients are being tested for responsiveness to a CAR-T cell therapy. In some embodiments, reference expression level can be the level in a sample previously obtained from the individual at a prior time. In some embodiments, the reference expression level cab be the level in a sample from a patient who received prior treatment with a CAR-T therapy but had a relapse of a health condition (e.g., relapse of LBCL). In some embodiments, the reference expression level cab be the level in a sample from a patient who received prior treatment with a CAR-T therapy and did not have a relapse of a health condition (e.g., LBCL). In some embodiments, the reference expression level cab be the level in a sample from a healthy individual. In some embodiments, individuals having a CD58 expression level that is less than a reference expression level, for instance a reference expression level in a sample from the groups as described above, can be identified as subjects/patients likely to be less responsive to treatment with a CAR-T therapy that does not comprise a trans CAR construct as described herein, such as a trans CAR construct comprising an anti-CD19 single chain variable fragment (scFv) FMC63 fused to a transmembrane domain and a CD2 intracellular domain. For example, such subjects/patients who exhibit CD58 expression levels that are about 90%, 80%, 70%, 60%, 50%, 45%, 40%, 35%, 30%, 25%, 20%, 15%, 10%, or 5% lower, relative to a reference CD58 expression level (such as the median level, noted above), or have a complete loss of expression of CD58, or can be identified as subjects/patients likely to respond to treatment with a trans CAR construct as described herein, such as a trans CAR construct comprising an anti-CD19 single chain variable fragment (scFv) FMC63 fused to a transmembrane domain and a CD2 intracellular domain.

As used herein, the phrase “one or more molecular alterations” refers to any variation in the genetic or protein sequence in or more cells of an individual as compared to the corresponding wild-type genes or proteins. The one or more molecular alterations can include, but are not limited to, genetic mutations, gene amplifications, splice variants, deletions, insertions/deletions (In/Del), gene rearrangements, single-nucleotide variations (SNVs), insertions, and aberrant RNA/protein expression. For example, in some embodiments, the molecular alterations in CD58 activity can include, an increased RNA/protein expression, a reduced RNA/protein expression, a loss of expression, an aberrant RNA/protein expression, a single nucleotide point mutation (SNP), a single-nucleotide variation (SNV), a gene amplification, a gene rearrangement, a gene fusion, a deletion, a frameshift deletion, an insertion, an InDel mutation, an epigenetic alteration, an amino acid substitution, and combinations of any thereof. In some embodiments, the one or more molecular alterations includes a loss of CD58 expression, a reduced expression of CD58 compared to a reference expression level of CD58, or expression of a mutated form of CD58. In some embodiments, at least one of the molecular alterations includes an amino acid substitution at a position corresponding to K60 of SEQ ID NO: 1 or SEQ ID NO: 2. In some embodiments, at least one of the molecular alterations includes an amino acid substitution at a position corresponding to K60 of SEQ ID NO: 1. In some embodiments, at least one of the molecular alterations includes an amino acid substitution at a position corresponding to K60 of SEQ ID NO: 2.

Non-limiting exemplary embodiments of the disclosed methods can include one or more of the following features. In some embodiments, the methods include administering a therapeutically effective amount of a CAR-T cell therapy to an individual who has, who is suspected of having, or who may be at high risk for developing one or more health conditions, such as proliferative diseases (e.g., cancers) associated with a decreased level or loss of CD58 expression or one or more molecular alterations in CD58 activity. In some embodiments, the individual is a patient under the care of a physician. Exemplary proliferative diseases can include, without limitation, angiogenic diseases, a metastatic diseases, tumorigenic diseases, neoplastic diseases and cancers. In some embodiments, the proliferative disease is a cancer. In some embodiments, the cancer is a pediatric cancer. In some embodiments, the cancer is a pancreatic cancer, a colon cancer, an ovarian cancer, a prostate cancer, a lung cancer, mesothelioma, a breast cancer, a urothelial cancer, a liver cancer, a head and neck cancer, a sarcoma, a cervical cancer, a stomach cancer, a gastric cancer, a melanoma, a uveal melanoma, a cholangiocarcinoma, multiple myeloma, leukemia, lymphoma, and glioblastoma.

In some embodiments, the health condition is a proliferative disorder selected from the group consisting of a solid tumor cancer, a non-solid tumor cancer, and a hematological malignancy. Exemplary cancers include, but are not limited to, large B-cell lymphoma (LBCL), B-cell acute lymphocytic leukemia (B-ALL), T-cell acute lymphocytic leukemia (T-ALL), acute lymphocytic leukemia (ALL), chronic myelogenous leukemia (CML), chronic lymphocytic leukemia (CLL), B cell promyelocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma (MCL), marginal zone lymphoma, multiple myeloma, myelodysplasia and myelodysplasia syndrome, B cell non-Hodgkin's lymphoma (NHL), Hodgkin's lymphoma (HL), plasmablastic lymphoma, plasmacytoid dendritic cell neoplasm, and Waldenstrom macroglobulinemia. In one embodiment, the cancer is ALL. In another embodiment, the cancer is CLL. In an embodiment, the cancer is associated with CD 19 expression.

In some embodiments, the cancer is a B-cell malignancy selected from the group consisting of non-Hodgkin's lymphoma, Burkitt's lymphoma, small lymphocytic lymphoma, large B-cell lymphoma (LBCL), primary effusion lymphoma, diffuse large B-cell lymphoma, splenic marginal zone lymphoma, MALT (mucosa-associated lymphoid tissue) lymphoma, hairy cell leukemia, chronic lymphocytic leukemia, B-cell prolymphocytic leukemia, B cell lymphomas (e.g. various forms of Hodgkin's disease, B cell non-Hodgkin's lymphoma (NHL), leukemias, acutelymphoblastic leukemia (ALL), chronic lymphocytic leukemia (CLL), B cell chronic lymphocytic leukemia (BCLL), hairy cell leukemia, chronic myoblastic leukemia, and myelomas.

In some embodiments, the one or more molecular alterations in CD58 activity is selected from the group consisting of an increased RNA/protein expression, a reduced RNA/protein expression, a loss of expression, an aberrant RNA/protein expression, a single nucleotide point mutation (SNP), a single-nucleotide variation (SNV), a gene amplification, a gene rearrangement, a gene fusion, a deletion, a frameshift deletion, an insertion, an InDel mutation, an epigenetic alteration, an amino acid substitution, and combinations of any thereof. In some embodiments, the one or more molecular alterations includes a loss of CD58 expression, a reduced expression of CD58 compared to a reference expression level of CD58, or expression of a mutated form of CD58. In some embodiments, the one or more molecular alterations includes an amino acid substitution at a position corresponding to K60 of SEQ ID NO: 1 or SEQ ID NO: 2. In some embodiments, the one or more molecular alterations includes an amino acid substitution at a position corresponding to K60 of SEQ ID NO: 1. In some embodiments, the one or more molecular alterations includes an amino acid substitution at a position corresponding to K60 of SEQ ID NO: 2. In some embodiments, the amino acid substitution is a Lys-to-Glu substitution (K60E). In some embodiments, the one or more molecular alterations includes an amino acid substitution at a position corresponding to C187 of SEQ ID NO: 1 or SEQ ID NO: 2. In some embodiments, the one or more molecular alterations includes an amino acid substitution at a position corresponding to C187 of SEQ ID NO: 1. In some embodiments, the one or more molecular alterations includes an amino acid substitution at a position corresponding to C187 of SEQ ID NO: 2. In some embodiments, the amino acid substitution is a Cys-to-Arg substitution (C187R).

In principle, there are no particular restrictions in regard to the types of biological samples suitable for use in the methods described herein. In some embodiments, the biological sample includes sputum, bronchoalveolar lavage, pleural effusion, tissue, whole blood, serum, plasma, buccal scrape, saliva, cerebrospinal fluid, urine, stool, circulating tumor cells, circulating nucleic acids, bone marrow, or any combination thereof. In some embodiments, the biological sample includes cells or tissue. For example, the biological sample can be a tissue sample, such as a biopsy, core biopsy, needle aspirate, or fine needle aspirate. In some embodiments, the biological sample can be a fluid sample, such as a blood sample, urine sample, or saliva sample. In some embodiments, the biological sample can be a skin sample. In some embodiments, the biological sample can be a cheek swab. In some embodiments, the biological sample includes whole blood and blood components. In some embodiments, the blood components include plasma. In some embodiments, the biological sample can be a plasma sample or serum sample. In some embodiments, the tissue is a tumor tissue or cancer tissue. In some embodiments, the biological sample includes tumor cells. In some embodiments, the biological sample is derived from a solid tumor, a soft tissue tumor, a non-solid tumor, a metastatic lesion, a circulating tumor cell (CTC) population. The biological sample can include an intact tissue sample. The biological sample can be a tumor cell line or derived from a xenograft model or patient derived xenograft (PDX). In some embodiments, the first and the second tumor samples are derived from the different subjects.

The interaction between the detection reagent with the CD58 encoding gene or a product thereof can be detected using one or more nucleic-acid-based analytical assays, protein-based analytical assay, or a combination thereof. Non-limiting examples of detection reagents suitable for the methods and systems of the disclosure include a double-stranded nucleic acids, a single-stranded nucleic acids (e.g., primers, probes), non-fluorescent and fluorescent nucleic acid-specific dyes, enzymes, and antibodies.

In some embodiments, the assessment of the presence and/or absence of one or more molecular alterations of a CD58-encoding gene or a product thereof and/or the detection of the interaction between the detection reagent with the CD58 encoding gene or a product thereof includes a nucleic-acid-based analytical assay selected from the group consisting of cancer personalized profiling by deep sequencing (CAPP-seq), nucleic acid sequencing, circulating tumor nucleic acid assessment, next generation sequencing (NGS), nucleic acid amplification-based assays, loop-mediated isothermal amplification (LAMP), rolling circle amplification (RCA), polymerase chain reaction (PCR), real-time PCR, quantitative reverse transcription PCR (qRT-PCR), PCR-RFLP assay, HPLC, mass-spectrometric genotyping, nucleic acid hybridization assay, comparative genomic hybridization, fluorescent in-situ hybridization (FISH), restriction digestion, capillary electrophoresis, and combinations of any thereof.

In some embodiments, the assessment of the presence and/or absence of one or more molecular alterations of a CD58-encoding gene includes cancer personalized profiling by deep sequencing (CAPP-seq) (see, e.g., Example 1). CAPP-seq is a next-generation sequencing based method used to analyze and/or quantify circulating tumor DNA in cancer (ctDNA). This method can be used for any cancer type that is known to have recurrent mutations. CAPP-Seq can detect one molecule of mutant DNA in 10,000 molecules of healthy DNA. The use of ctDNA in this technique should not be confused with circulating tumor cells (CTCs).

In some embodiments, an electrophoretic mobility assay is used to acquire the knowledge of the one or more molecular alterations in CD58 activity present in the biological sample obtained from an individual. For example, a nucleic acid sequence encoding a mutation can be detected by amplifying the nucleic acid region corresponding to the one or more alterations in a CD58 gene and comparing the electrophoretic mobility of the amplified nucleic acid to the electrophoretic mobility of the corresponding region in a wild-type CD58 gene.

In some embodiments, the analytical assay used to acquire the knowledge of the one or more molecular alterations in CD58 activity present in the biological sample involves a nucleic acid hybridization assay that includes contacting nucleic acids derived from the biological sample with a nucleic acid probe comprising (1) a nucleic acid sequence complementary to a nucleic acid sequence encoding the one or more mutations and further comprising (2) a detectable label.

In some embodiments, the analytical assay used to acquire the knowledge of the one or more molecular alterations in CD58 activity present in the biological sample involves polymerase chain reactions (PCR) or nucleic acid amplification-based assays. A number of PCR-based analytical assays known in the art are suitable for the methods disclosed herein, comprising but not limited to real-time PCR, quantitative reverse transcription PCR (qRT-PCR), PCR-RFLP assay, loop-mediated isothermal amplification (LAMP), and rolling circle amplification (RCA).

In some embodiments, the analytical assay used to acquire the knowledge of the one or more molecular alterations in CD58 activity present in the biological sample involves determining a nucleic acid sequence and/or an amino acid sequence comprising the one or more molecular alterations. In some embodiments, the nucleic acid sequence comprising the one or more molecular alterations from a cancer patient is sequenced. In some embodiments, the sequence is determined by a next generation sequencing procedure. As used herein “next-generation sequencing” refers to oligonucleotide sequencing technologies that have the capacity to sequence oligonucleotides at speeds above those possible with conventional sequencing methods (e.g. Sanger sequencing), due to performing and reading out thousands to millions of sequencing reactions in parallel. Non-limiting examples of next-generation sequencing methods/platforms include Massively Parallel Signature Sequencing (Lynx Therapeutics); solid-phase, reversible dye-terminator sequencing (Solexa/Illumina); DNA nanoball sequencing (Complete Genomics); SOLiD technology (Applied Biosystems); 454 pyro-sequencing (454 Life Sciences/Roche Diagnostics); ion semiconductor sequencing (ION Torrent); and technologies available from Pacific Biosciences, Intelligen Bio-systems, Oxford Nanopore Technologies, and Helicos Biosciences.

Accordingly, in some embodiments, the NGS procedure used in the methods disclosed herein can comprise pyrosequencing, sequencing by synthesis, sequencing by ligation, or a combination of any thereof. In some embodiments, the NGS procedure is performed by an NGS platform selected from Illumina, Ion Torrent, Qiagen, Invitrogen, Applied Biosystem, Helicos, Oxford Nanopore, Pacific Biosciences, and Complete Genomics.

In some embodiments, FISH analysis can be used to identify the chromosomal mutations resulting in the one or more molecular alterations such as the mutated genes or mutated gene products (i.e., CD58 polypeptides) as described herein. For example, to perform FISH, at least a first probe tagged with a first detectable label can be designed to target a mutated gene of a mutated polypeptide, and at least a second probe tagged with a second detectable label can be designed to target the corresponding wild-type gene or wild-type polypeptide such that one of ordinary skill in the art observing the probes can determine that a relevant gene or gene product is present in the sample. Generally, FISH assays are performed using formalin-fixed, paraffin-embedded tissue sections that are placed on slides. For example, the DNA from the biological samples is denatured to single-stranded form and subsequently allowed to hybridize with the appropriate DNA probes that can be designed and prepared using methods and techniques known to those having ordinary skill in the art. Following hybridization, any unbound probe may be removed by a series of washes and the nuclei of the cells are counter-stained with DAPI (4′,6 diamidino-2-phenylindole), a DNA-specific stain that fluoresces blue. Hybridization of the probe or probes are viewed using a fluorescence microscope equipped with appropriate excitation and emission filters, allowing visualization of the fluorescent signals. Other variations of the FISH method known in the art are also suitable for evaluating an individual selected in accordance with the methods disclosed herein.

In some embodiments, the assessment of the presence and/or absence of one or more molecular alterations of a CD58-encoding gene or a product thereof and/or the detection of the interaction between the detection reagent with the CD58 encoding gene or a product thereof includes a protein-based analytical assay selected from the group consisting of immunohistochemistry (IHC), protein-microarray, western blotting, mass spectrometry, flow cytometry, enzyme-linked immunosorbent assay (ELISA), immunofluorescence staining, multiplex detection assay, and combinations of any thereof. In some embodiments, the protein-based assay includes the use of one or more antibodies that selectively bind to one or more of wild-type CD58 or mutated CD58 polypeptides. Exemplary CD58 monoclonal and polyclonal antibodies useful for protein-based analytical assays include those made commercially available by Abcam (Cat #ab196648, ab275392, ab281201, and ab91058), LSBio (Cat #LS-C819068-50), and Thermo Fischer Scientific (Cat #MA5800, MA5-29120, and MA5-29121). In some embodiments, the assessment of the presence and/or absence of one or more molecular alterations of a CD58-encoding gene or a product thereof include immunohistochemistry (IHC) (see, e.g., Example 1).

In some embodiments, the one or more molecular alterations in the CD58-encoding gene or a product thereof reduces binding affinity of a CD58 protein product to its ligand CD2.

The term “binding affinity” is herein used as a measure of the strength of a non-covalent interaction between two molecules, e.g., a polypeptide and its ligand. The term “binding affinity” is used to describe monovalent interactions (intrinsic activity). Binding affinity between two molecules may be quantified by determination of the dissociation constant (K_{D}). In turn, K_{D} can be determined by measurement of the kinetics of complex formation and dissociation using, e.g., the surface plasmon resonance (SPR) method (Biacore). The rate constants corresponding to the association and the dissociation of a monovalent complex are referred to as the association rate constants kₐ (or kₒₙ) and dissociation rate constant k_{d} (or k_{off}), respectively. K_{D} is related to kₐ and k_{d} through the equation K_{D}=k_{d}/kₐ. The value of the dissociation constant can be determined directly by well-known methods, and can be computed even for complex mixtures by methods such as those set forth in Caceci et al. (1984, Byte 9: 340-362). For example, the K_{D} may be established using a double-filter nitrocellulose filter binding assay such as that disclosed by Wong & Lohman (1993, Proc. Natl. Acad. Sci. USA 90: 5428-5432). Other standard assays to evaluate the binding ability of antibodies or polypeptides of the present disclosure towards target antigens are known in the art, including for example, ELISAs, Western blots, RIAs, and flow cytometry analysis, and other assays exemplified elsewhere herein. The binding kinetics and binding affinity of the polypeptide to its ligand also can be assessed by standard assays known in the art, such as Surface Plasmon Resonance (SPR), e.g., by using a Biacore™ system, or KinExA.

In some embodiments, the methods of the disclosure further include treating the health condition with a CAR-T cell therapy. In some embodiments, the CAR-T cell therapy is administered to individual as single therapy or in combination with one or more additional therapies. In some embodiments, the CAR-T cell therapy and/or at least one additional therapy includes a CAR construct including a CD2 costimulatory domain. In some embodiments, the CAR construct including a CD2 costimulatory domain comprises the amino acid sequence of SEQ ID NO: 4.

In some embodiments, the CAR-T cell therapy targets an antigen that is expressed at low density on target cells, e.g., less than about 6,000 molecules of the target antigen per cell. In some embodiments, the antigen is expressed at a density of less than about 5,000 molecules, less than about 4,000 molecules, less than about 3,000 molecules, less than about 2,000 molecules, less than about 1,000 molecules, or less than about 500 molecules of the target antigen per cell. In some embodiments, the antigen is expressed at a density of less than about 2,000 molecules, such as e.g., less than about 1,800 molecules, less than about 1,600 molecules, less than about 1,400 molecules, less than about 1,200 molecules, less than about 1,000 molecules, less than about 800 molecules, less than about 600 molecules, less than about 400 molecules, less than about 200 molecules, or less than about 100 molecules of the target antigen per cell. In some embodiments, the antigen is expressed at a density of less than about 1,000 molecules, such as e.g., less than about 900 molecules, less than about 800 molecules, less than about 700 molecules, less than about 600 molecules, less than about 500 molecules, less than about 400 molecules, less than about 300 molecules, less than about 200 molecules, or less than about 100 molecules of the target antigen per cell. In some embodiments, the antigen is expressed at a density ranging from about 5,000 to about 100 molecules of the target antigen per cell, such as e.g., from about 5,000 to about 1,000 molecules, from about 4,000 to about 2,000 molecules, from about 3,000 to about 2,000 molecules, from about 4,000 to about 3,000 molecules, from about 3,000 to about 1,000 molecules, from about 2,000 to about 1,000 molecules, from about 1,000 to about 500 molecules, from about 500 to about 100 molecules of the target antigen per cell.

Administration of any one of the CAR-T cell therapies described herein, e.g., engineered CAR-T cells can be used to treat patients in the treatment of relevant health conditions, such as proliferative diseases (e.g., cancers), autoimmune diseases, and microbial infections (e.g., viral infections). In some embodiments, one or more engineered CAR-T cells as described herein can be incorporated into therapeutic agents for use in methods of treating an individual who has, who is suspected of having, or who may be at high risk for developing one or more health conditions, such as proliferative diseases (e.g., cancers), autoimmune diseases, and chronic infections. In some embodiments, the individual is a patient under the care of a physician.

In some embodiments, the methods include calculating or administering a therapeutically effective amount of a CAR-T cell therapy to an individual in need thereof. The term “effective amount”, “therapeutically effective amount”, or “pharmaceutically effective amount” of an engineered CAR-T cell generally refers to an amount or number sufficient for a population of engineered CAR-T cells or a pharmaceutical composition to accomplish a stated purpose relative to the absence of the engineered cell population or pharmaceutical composition (e.g., achieve the effect for which it is administered, treat a disease, reduce a signaling pathway, or reduce one or more symptoms of a disease or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a T-cell population or composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

Exemplary proliferative diseases can include, without limitation, angiogenic diseases, a metastatic diseases, tumorigenic diseases, neoplastic diseases and cancers. In some embodiments, the proliferative disease is a cancer. In some embodiments, the cancer is a pediatric cancer. In some embodiments, the cancer is a pancreatic cancer, a colon cancer, an ovarian cancer, a prostate cancer, a lung cancer, mesothelioma, a breast cancer, a urothelial cancer, a liver cancer, a head and neck cancer, a sarcoma, a cervical cancer, a stomach cancer, a gastric cancer, a melanoma, a uveal melanoma, a cholangiocarcinoma, multiple myeloma, leukemia, lymphoma, and glioblastoma.

In some embodiments, the health condition is a proliferative disorder selected from the group consisting of a solid tumor cancer, a non-solid tumor cancer, and a hematological malignancy. Exemplary cancers include, but are not limited to, large B-cell lymphoma (LBCL), B-cell acute lymphocytic leukemia (B-ALL), T-cell acute lymphocytic leukemia (T-ALL), acute lymphocytic leukemia (ALL), chronic myelogenous leukemia (CML), chronic lymphocytic leukemia (CLL), B cell promyelocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma (MCL), marginal zone lymphoma, multiple myeloma, myelodysplasia and myelodysplasia syndrome, B cell non-Hodgkin's lymphoma (NHL), Hodgkin's lymphoma (HL), plasmablastic lymphoma, plasmacytoid dendritic cell neoplasm, and Waldenstrom macroglobulinemia. In one embodiment, the cancer is ALL. In another embodiment, the cancer is CLL. In an embodiment, the cancer is associated with CD 19 expression.

In some embodiments, the cancer is a B-cell malignancy selected from the group consisting of non-Hodgkin's lymphoma, Burkitt's lymphoma, small lymphocytic lymphoma, large B-cell lymphoma (LBCL), primary effusion lymphoma, diffuse large B-cell lymphoma, splenic marginal zone lymphoma, MALT (mucosa-associated lymphoid tissue) lymphoma, hairy cell leukemia, chronic lymphocytic leukemia, B-cell prolymphocytic leukemia, B cell lymphomas (e.g. various forms of Hodgkin's disease, B cell non-Hodgkin's lymphoma (NHL), leukemias, acutelymphoblastic leukemia (ALL), chronic lymphocytic leukemia (CLL), B cell chronic lymphocytic leukemia (BCLL), hairy cell leukemia, chronic myoblastic leukemia, and myelomas.

In some embodiments, the cancer is a multiply drug resistant cancer or a recurrent cancer. It is contemplated that the compositions and methods disclosed here are suitable for both non-metastatic cancers and metastatic cancers. Accordingly, in some embodiments, the cancer is a non-metastatic cancer. In some other embodiments, the cancer is a metastatic cancer. In some embodiments, the composition administered to the individual inhibits metastasis of the cancer in the individual. In some embodiments, the administered CAR-T cell therapy inhibits tumor growth in the individual.

For example, in some embodiments, the CAR-T cell therapy administered to the individual can reduce metastatic nodules in the individual. In some embodiments, the administered CAR-T cell therapy inhibits tumor growth in the individual.

In some embodiments, the administered CAR-T cells inhibit proliferation of a target cancer cell, and/or inhibits tumor growth of the cancer in the individual. For example, the target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the CAR-T cells described herein, wherein the administered CAR-T cells inhibit the proliferation of the target cell and/or inhibit tumor growth of a target cancer in the individual compared to the proliferation of the target cell and/or tumor growth of the target cancer in subjects who have not been administered with the CAR-T cell therapy.

Administration of the CAR-T cell therapies described herein, e.g., engineered CAR-T cells, can be used in the stimulation of an immune response. In some embodiments, one or more of engineered CAR-T cells as described herein are administered to an individual after induction of remission of cancer with chemotherapy, or after autologous or allogeneic hematopoietic stem cell transplantation. In some embodiments, compositions described herein are administered to an individual in need of increasing the production of interferon gamma (IFNγ), tumor-necrosis factor alpha (TNFα), and/or interleukin-2 (IL-2) in the treated subject relative to the production of these molecules in subjects who have not been administered one of the therapeutic compositions disclosed herein.

An effective amount of the CAR-T cell therapies described herein, e.g., engineered CAR-T cells, can be determined based on the intended goal, for example cancer regression. For example, where existing cancer is being treated, the amount of a composition disclosed herein to be administered may be greater than where administration of the composition is for prevention of cancer. One of ordinary skill in the art would be able to determine the amount of a composition to be administered and the frequency of administration in view of this disclosure. The quantity to be administered, both according to number of treatments and dose, also depends on the individual to be treated, the state of the individual, and the protection desired. Precise amounts of the composition also depend on the judgment of the practitioner and are peculiar to each subject. Frequency of administration could range from 1-2 days, to 2-6 hours, to 6-10 hours, to 1-2 weeks or longer depending on the judgment of the practitioner.

Determination of the amount of compositions to be administered will be made by one of skill in the art, and will in part be dependent on the extent and severity of cancer, and whether the engineered CAR-T cells are being administered for treatment of existing cancer or prevention of cancer. For example, longer intervals between administration and lower amounts of compositions may be employed where the goal is prevention. For instance, amounts of compositions administered per dose may be 50% of the dose administered in treatment of active disease, and administration may be at weekly intervals. One of ordinary skill in the art, in light of this disclosure, would be able to determine an effective amount of compositions and frequency of administration. This determination would, in part, be dependent on the particular clinical circumstances that are present (e.g., type of cancer, severity of cancer).

In some embodiments, it may be desirable to provide a continuous supply of a composition disclosed herein to the individual to be treated, e.g., a patient. In some embodiments, continuous perfusion of the region of interest (such as a tumor) may be suitable. The time period for perfusion would be selected by the clinician for the particular subject and situation, but times could range from about 1-2 hours, to 2-6 hours, to about 6-10 hours, to about 10-24 hours, to about 1-2 days, to about 1-2 weeks or longer. Generally, the dose of the composition via continuous perfusion will be equivalent to that given by single or multiple injections, adjusted for the period of time over which the doses are administered.

In some embodiments, administration is by intravenous infusion. An effective amount of the engineered CAR-T cells described herein can be determined based on the intended goal, for example tumor regression. For example, where existing cancer is being treated, the number of cells to be administered may be greater than where administration of the engineered CAR-T cells disclosed herein is for prevention of cancer. One of ordinary skill in the art would be able to determine the number of cells to be administered and the frequency of administration in view of this disclosure. The quantity to be administered, both according to number of treatments and dose, also depends on the individual to be treated, the state of the individual, and the protection desired. Precise amounts of the therapeutic composition also depend on the judgment of the practitioner and are peculiar to each individual. Frequency of administration could range from 1-2 days, to 2-6 hours, to 6-10 hours, to 1-2 weeks or longer depending on the judgment of the practitioner. Generally, the dose of the therapeutic composition via continuous perfusion will be equivalent to that given by single or multiple injections, adjusted for the period of time over which the doses are administered.

### Additional Therapies

As discussed above, any one of the CAR-T cell therapies as described herein, e.g., can be administered to an individual in need thereof as a single therapy (e.g., monotherapy). In addition or alternatively, in some embodiments of the disclosure, one or more of the CAR-T cell therapies described herein can be administered to the individual in combination with one or more additional therapies, e.g., at least one, two, three, four, or five additional therapies. Suitable therapies to be administered in combination with the CAR-T cell therapies described herein include, but are not limited to chemotherapy, radiotherapy, immunotherapy, hormonal therapy, toxin therapy, targeted therapy, and surgery. Other suitable therapies include therapeutic agents such as chemotherapeutics, anti-cancer agents, and anti-cancer therapies.

Due to the involvement of the CD2/CD58 pathway in cellular processes associated with immune responses, tumorigenesis and other disease states, any one of the CAR-T cell therapies as described herein, e.g., can be administered to an individual in need thereof with one or more therapeutic agents that target this pathway. For examples, molecules that modulate CD2 activity can be immunosuppressive agents and/or anti-inflammatory agents and/or anticancer agents with activity towards (1) autoimmune disorders such as multiple sclerosis; (2) a variety of inflammatory diseases or disorders with an inflammatory or T cell-mediated component such as various forms of arthritis; allograft rejections; asthma; inflammatory diseases of the bowel, including Crohn's disease; various dermatological conditions such as psoriasis; and the like, and (3) a variety of cancers and tumors.

Administration “in combination with” one or more additional therapies includes simultaneous (concurrent) and consecutive administration in any order. In some embodiments, the one or more additional therapies is selected from the group consisting of chemotherapy, radiotherapy, immunotherapy, hormonal therapy, toxin therapy, and surgery. The term chemotherapy as used herein encompasses anti-cancer agents. Various classes of anti-cancer agents can be suitably used for the methods disclosed herein. Non-limiting examples of anti-cancer agents include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

Topoisomerase inhibitors are also another class of anti-cancer agents that can be used herein. Topoisomerases may be essential enzymes that maintain the topology of DNA. Inhibition of type I or type II topoisomerases interferes with both transcription and replication of DNA by upsetting proper DNA supercoiling. Some type I topoisomerase inhibitors include camptothecins such as irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple (Podophyllum peltatum).

Antineoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell's DNA.

Alkylating agents can alkylate many nucleophilic functional groups under conditions present in cells. Cisplatin and carboplatin, and oxaliplatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinblastine, vinorelbine, and vindesine.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are obtained from plants and block cell division by preventing microtubule function. Since microtubules are vital for cell division, without them, cell division cannot occur in some instances. The main examples are vinca alkaloids and taxanes.

Podophyllotoxin is a plant-derived compound which has been reported to help with digestion as well as used to produce two other cytostatic drugs, etoposide and teniposide. They prevent the cell from entering the G1 phase (the start of DNA replication) and the replication of DNA (the S phase).

Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

In some embodiments, the anti-cancer agents can be selected from remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, gefitinib (Iressa®), taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib (Velcade®), bisphosphonate, arsenic trioxide, vincristine, doxorubicin (Doxil®), paclitaxel, ganciclovir, adriamycin, estrainustine sodium phosphate (Emcyt®), sulindac, etoposide, and combinations of any thereof.

In other embodiments, the anti-cancer agent can be selected from bortezomib, cyclophosphamide, dexamethasone, doxorubicin, interferon-alpha, lenalidomide, melphalan, pegylated interferon-alpha, prednisone, thalidomide, or vincristine.

In some embodiments, the methods of treatment as described herein further include an immunotherapy. In some embodiments, the immunotherapy includes administration of one or more checkpoint inhibitors. Accordingly, some embodiments of the methods of treatment described herein include further administration of a compound that inhibits one or more immune checkpoint molecules. Non-limiting examples of immune checkpoint molecules include CTLA4, PD-1, PD-L1, A2AR, B7-H3, B7-H4, TIM3, and combinations of any thereof. In some embodiments, the compound that inhibits the one or more immune checkpoint molecules includes an antagonistic antibody. Examples of antagonistic antibodies suitable for the compositions and methods disclosed herein include, but are not limited to, ipilimumab, nivolumab, pembrolizumab, durvalumab, atezolizumab, tremelimumab, and avelumab.

In some aspects, the one or more anti-cancer therapy is radiation therapy. In some embodiments, the radiation therapy can include the administration of radiation to kill cancerous cells. Radiation interacts with molecules in the cell such as DNA to induce cell death. Radiation can also damage the cellular and nuclear membranes and other organelles. Depending on the radiation type, the mechanism of DNA damage may vary as does the relative biologic effectiveness. For example, heavy particles (i.e. protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray. Radiation also contemplated herein includes, for example, the directed delivery of radioisotopes to cancer cells. Other forms of DNA damaging factors are also contemplated herein such as microwaves and UV irradiation.

Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a fractioned regime. For example, the regime may include fractionated individual doses of 2 Gy. Dosage ranges for radioisotopes vary widely, and depends on the half-life of the isotope and the strength and type of radiation emitted. When the radiation includes use of radioactive isotopes, the isotope may be conjugated to a targeting agent, such as a therapeutic antibody, which carries the radionucleotide to the target tissue (e.g., tumor tissue).

Surgery described herein includes resection in which all or part of a cancerous tissue is physically removed, exercised, and/or destroyed. Tumor resection refers to physical removal of at least part of a tumor. In addition to tumor resection, treatment by surgery includes laser surgery, cryosurgery, electrosurgery, and microscopically controlled surgery (Mohs surgery). Removal of pre-cancers or normal tissues is also contemplated herein.

Accordingly, in some embodiments, the methods of the disclosure include administration of a CAR-T cell therapy described herein to a subject individually as a single therapy (e.g., monotherapy). In some embodiments, a CAR-T cell therapy of the disclosure is administered to an individual as a first therapy in combination with a second therapy. In some embodiments, the second therapy is selected from the group consisting of chemotherapy, radiotherapy, immunotherapy, hormonal therapy, toxin therapy, and surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Administration of CAR T Cells to an Individual

In some embodiments, the methods of the disclosure involve administering an effective amount or number of the engineered CAR-T cells described here to an individual in need thereof. This administering step can be accomplished using any method of implantation delivery in the art. For example, the engineered CAR-T cells can be infused directly in the individual's bloodstream or otherwise administered to the individual.

In some embodiments, the methods disclosed herein include administering, which term is used interchangeably with the terms “introducing,” implanting,” and “transplanting,” engineered CAR-T cells into an individual, by a method or route that results in at least partial localization of the introduced cells at a desired site such that a desired effect(s) is/are produced. The engineered CAR-T cells or their differentiated progeny can be administered by any appropriate route that results in delivery to a desired location in the individual where at least a portion of the administered cells or components of the cells remain viable. The period of viability of the cells after administration to an individual can be as short as a few hours, e.g., twenty-four hours, to a few days, to as long as several years, or even the lifetime of the individual, e.g., long-term engraftment.

When provided prophylactically, the engineered CAR-T cells described herein can be administered to an individual in advance of any symptom of a disease or health condition to be treated. Accordingly, in some embodiments the prophylactic administration of an engineered CAR-T cell population prevents the occurrence of symptoms of the disease or health condition.

When provided therapeutically in some embodiments, engineered CAR-T cells are provided at (or after) the onset of a symptom or indication of a disease or health condition, e.g., upon the onset of disease or health condition.

For use in the various embodiments described herein, an effective amount of engineered CAR-T cells as described herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof.

In some embodiments, the engineered CAR-T cells are non-autologous to the individual in need of treatment. In some embodiments, the adoptive cell therapy is an allogeneic adoptive cell therapy. For example, in some embodiments, the engineered CAR-T cells are allogeneic to the individual in need of treatment. In an allogeneic adoptive cell therapy, the engineered CAR-T cells are not derived from the individual receiving the adoptive cell therapy. Allogeneic cell therapy generally refers to a therapy whereby the individual (donor) who provides the T cells is a different individual (of the same species) than the individual receiving the cell therapy. For example, a population of engineered CAR-T cells being administered to an individual is derived from one more unrelated donors, or from one or more non-identical siblings. Accordingly, the engineered CAR-T cells can be derived from one or more donors or can be obtained from an autologous source. In some embodiments, the engineered CAR-T cells are expanded in culture prior to administration to an individual in need thereof.

In some embodiments, the delivery of a cell composition (e.g., a composition including a plurality of engineered CAR-T cells described herein) into an individual by a method or route results in at least partial localization of the cell composition at a desired site. A composition including engineered CAR-T cells can be administered by any appropriate route that results in effective treatment in the individual, e.g., administration results in delivery to a desired location in the individual where at least a portion of the composition delivered, e.g., at least 1×10⁴ cells, is delivered to the desired site for a period of time. Modes of administration include injection, infusion, instillation. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is often considered a standard mode of administration.

In some embodiments, the engineered CAR-T cells are administered systemically, e.g., via infusion or injection. For example, a population of engineered CAR-T cells as described herein are administered other than directly into a target site, tissue, or organ, such that it enters, the individual's circulatory system and, thus, is subject to metabolism and other similar biological processes.

The efficacy of a treatment including any of the compositions provided herein for the prevention or treatment of a disease or health condition can be determined by a skilled clinician. However, one skilled in the art will appreciate that a prevention or treatment is considered effective if any one or all of the signs or symptoms or markers of disease are improved or ameliorated. Efficacy can also be measured by failure of an individual to worsen as assessed by decreased hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

Measurement of the degree of efficacy is based on parameters selected with regard to the disease being treated and the symptoms experienced. In general, a parameter is selected that is known or accepted as correlating with the degree or severity of the disease, such as a parameter accepted or used in the medical community. For example, in the treatment of a solid cancer, suitable parameters can include reduction in the number and/or size of metastases, number of months of progression-free survival, overall survival, stage or grade of the disease, the rate of disease progression, the reduction in diagnostic biomarkers (for example without limitation, a reduction in circulating tumor DNA or RNA, a reduction in circulating cell-free tumor DNA or RNA, and the like), and combinations thereof. It will be understood that the effective dose and the degree of efficacy will generally be determined with relation to a single subject and/or a group or population of subjects. Therapeutic methods of the disclosure reduce symptoms and/or disease severity and/or disease biomarkers by at least about 1, 2, 3, 4, 5, 10, 15, 20, 25, 30, 35, 40, 45, 50, 55, 60, 65, 70, 75, 80, 85, 90, 95, 96, 97, 98, 99, or 100%.

As discussed above, a therapeutically effective amount of a pharmaceutical composition can be an amount of the pharmaceutical composition that is sufficient to promote a particular beneficial effect when administered to an individual, such as one who has, is suspected of having, or is at risk for a disease or health condition. In some embodiments, an effective amount includes an amount sufficient to prevent or delay the development of a symptom of the disease or health condition, alter the course of a symptom of the disease or health condition (for example but not limited to, slow the progression of a symptom of the disease), or reverse a symptom of the disease or health condition. It is understood that for any given case, an appropriate effective amount can be determined by one of ordinary skill in the art using routine experimentation.

### Kits

Also provided herein are kits for the practice of one or more methods described herein, including methods for the diagnosis and/or treatment of a health condition in an individual. Generally, the kits of the disclosure can include (i) reagents for assessing the presence and/or absence of one or more molecular alterations of a CD58-encoding gene or a product thereof in a biological sample, and (ii) instructions for using the kits. For examples, some embodiments of the disclosure provide kits for determining the responsiveness of an individual to a CAR-T cell therapy. Some embodiments of the disclosure provide kits for identifying an individual who has an increased unresponsiveness to a CAR-T cell therapy. Some embodiments of the disclosure provide kits for optimizing the therapeutic efficacy of a CAR-T cell therapy in an individual. Some embodiments of the disclosure provide kits for administering a CAR-T cell therapy to an individual.

In some embodiments, the kits include a detection reagent for detecting interaction between the detection reagent with a CD58 encoding gene or a product thereof in a biological sample from the individual.

In some embodiments, the instructions for use provide that if at least one of the one or more molecular alterations in CD58 activity is detected in the sample, the individual is identified as having decreased responsiveness to treatment with the CAR-T cell therapy. In some embodiments, the instructions for use provide that (i) if at least one of the one or more molecular alterations in CD58 activity is detected in the sample, the individual is selected as having increased unresponsiveness to treatment with a CAR-T cell therapy; or (ii) none of the one or more molecular alterations in CD58 activity is detected in the sample, the individual is selected as not having increased unresponsiveness to treatment with the CAR-T cell therapy. In some embodiments, the instructions for use include instructions for identifying a therapeutically effective amount of the CAR-T cell therapy based on the detected interaction between the detection reagent with the CD58 encoding gene or a product thereof. In some embodiments, the instructions for use include instructions for administering a therapeutically effective amount of the CAR-T cell therapy based on the detected interaction between the detection reagent with the CD58 encoding gene or a product thereof.

In some embodiments, the kits may further comprise one or more of: extraction buffer/reagents and protocol, amplification buffer/reagents and protocol, hybridization buffer/reagents and protocol, and labeling buffer/reagents and protocol.

For example, any of the above-described kits can further include one or more additional reagents, where such additional reagents can be selected from: dilution buffers; reconstitution solutions, wash buffers, control reagents, control expression vectors, negative control T-cell populations, positive control T-cell populations, reagents for ex vivo production of the T-cell populations.

In some embodiments, the kits of the disclosure further include one or more syringes (including pre-filled syringes) and/or catheters (including pre-filled syringes) used to administer a CAR-T cell therapy to an individual in need thereof. In some embodiments, a kit can have one or more additional therapeutic agents that can be administered simultaneously or sequentially with the other kit components for a desired purpose, e.g., for inhibiting a target cancer cell, or for treating a health condition in an individual in need thereof.

In some embodiments, the components of a kit can be in separate containers. In some other embodiments, the components of a kit can be combined in a single container.

In some embodiments, a kit can further include instructions for using the components of the kit to practice the methods. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

### Genetic-Based Systems

The methods for diagnosis and treatment of health conditions as described herein can be implemented using a variety of hardware components. In this section, examples of such components are described. However, it should be understood that in general, the various steps and techniques discussed herein can be performed using a variety of different devices and system components, not all of which are expressly set forth.

In another aspect, some embodiments of the disclosure relate to systems for diagnosis and/or treatment of health conditions, the systems including: a) a logic processor; and b) a stored program code that is executable by the logic processor, which when executed by the processor provides operations for performing a method of diagnosis and/or treatment of health conditions according to present disclosure. In some embodiments, the systems include (a) a logic processor; and (b) a stored program code that is executable by the logic processor, which when executed by the processor provides operations for performing one or more of the following: (i) determining the responsiveness of an individual to CAR-T cell therapy; (ii) identifying an individual as having increased unresponsiveness to treatment with a CAR-T cell therapy; (iii) optimizing the therapeutic efficacy of a CAR-T cell therapy in an individual; and (iv) calculating or administering a therapeutically effective amount of a CAR-T cell therapy to an individual.

Non-limiting exemplary embodiments of the systems of the disclosure can include one or more of the following features. In some embodiments, the systems disclosed herein further include a report engine communicatively coupled to the logic processor, wherein reports produced by the report engine depend upon results from execution of the program code, wherein the program code configures the logic processor to receive a preselected set of data input pertaining to the presence and/or absence of one or more molecular alterations of a CD58-encoding gene or a product thereof in a biological obtained from an individual in order to assign a relative score to the individual's responsiveness to CAR-T cell therapy based at least in part on the preselected set of data input, and optionally: (a) determining the responsiveness of the individual to CAR-T cell therapy; (b) identifying the individual as having increased unresponsiveness to treatment with a CAR-T cell therapy; and/or (c) optimizing the therapeutic efficacy of a CAR-T cell therapy in the individual.

In some embodiments, the systems of the disclosure further include generating a report that contains information relevant to the individuals identified as having increased unresponsiveness to a CAR-T cell therapy and/or relevant to the CAR-T cell therapies identified as being effective for treatment of health conditions. In some embodiments, the profile report is characterized as having an encoding selected from the group consisting of “.doc”; “.pdf”; “.xml”; “.html”; “.jpg”; “.aspx”; “.php”, and a combination of any thereof.

In yet another aspect, provided herein is a non-transitory computer readable medium containing machine executable instructions that when executed cause a processor to perform operations including: receiving a report including a preselected set of data input; assigning, based at least in part on the report, a relative performance score to the identified CAR-T cell therapy; and outputting a report for the CAR-T cell therapy based upon the assigned performance score. In some embodiments, provided herein is a non-transitory computer readable medium containing machine executable instructions that when executed cause a processor to perform operations including: receiving a report including a preselected set of data input; assigning, based at least in part on the report, a relative unresponsiveness score to the identified individual; and outputting a report for the individual based upon the assigned unresponsiveness score. Accordingly, CAR-T cell therapy reports and individual reports generated by the systems of the disclosure are also with the scope of this disclosure.

Each of the aspects and embodiments described herein are capable of being used together, unless excluded either explicitly or clearly from the context of the embodiment or aspect.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

The experimental results described below demonstrate that LBCL patients whose tumors have mutated or lost expression of CD58 are unlikely to derive durable benefit from CD19 CAR-T cell therapy. In particular, CD58 mutations and protein loss have previously been described in LBCL and have been linked to both immune escape and poor patient outcomes. In some experiments described below, tumor microarrays were stained by IHC, followed by deep sequencing analysis of circulating tumor DNA to ascertain CD58 status. Neither method can detect all cases of CD58 alteration, which is also likely to be heterogeneous in certain patients. Direct sequencing of genomic DNA and RNA for CD58 from tumor samples could provide additional data to correlate with patient outcome, which was not available in the patient series tested in these studies. The patients in these studies s who had CD58 alterations were also more likely to be younger and have bulky disease with high low density lipoprotein (LDH). Additional experiments are being planned for large, prospective series to tease apart the competing contributions from these additional risk factors. Additionally, the experimental finding that CD58 is correlated to response is a retrospective finding from patients treated at a single institution. Though the experimental result described herein provides convincing evidence from xenograft models on the importance of CD58 expression for CAR functionality, this result can benefit from larger, prospective trials of LBCL patients undergoing CD19 CAR therapy.

### Example 1

CD58 May be Required for Durable Remission in LBCL Patients Receiving Treatment with Axicabtagene Ciloleucel

This Example describes the results of studies performed to illustrate that durable remission of in LBCL patients receiving a treatment of the FDA-approved drug axicabtagene ciloleucel (YESCARTA®) requires CD58 expression.

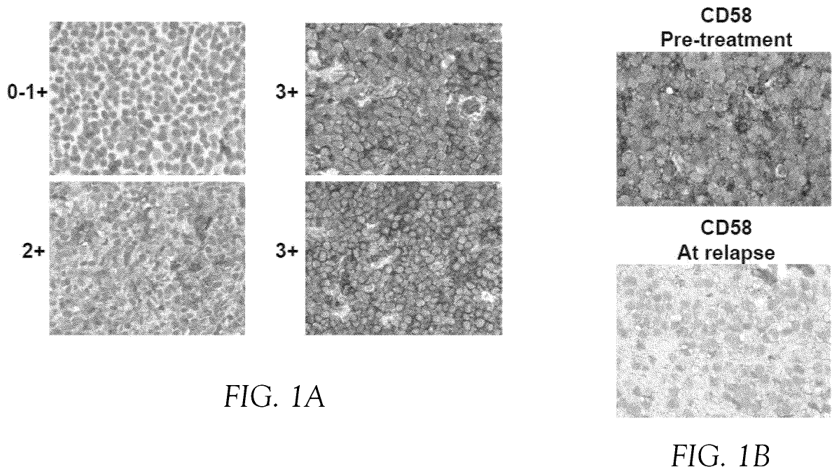

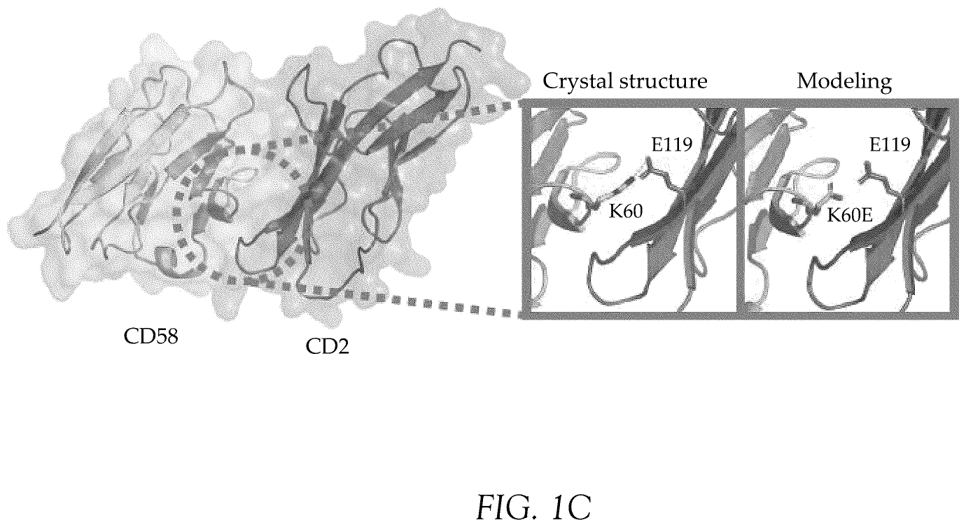

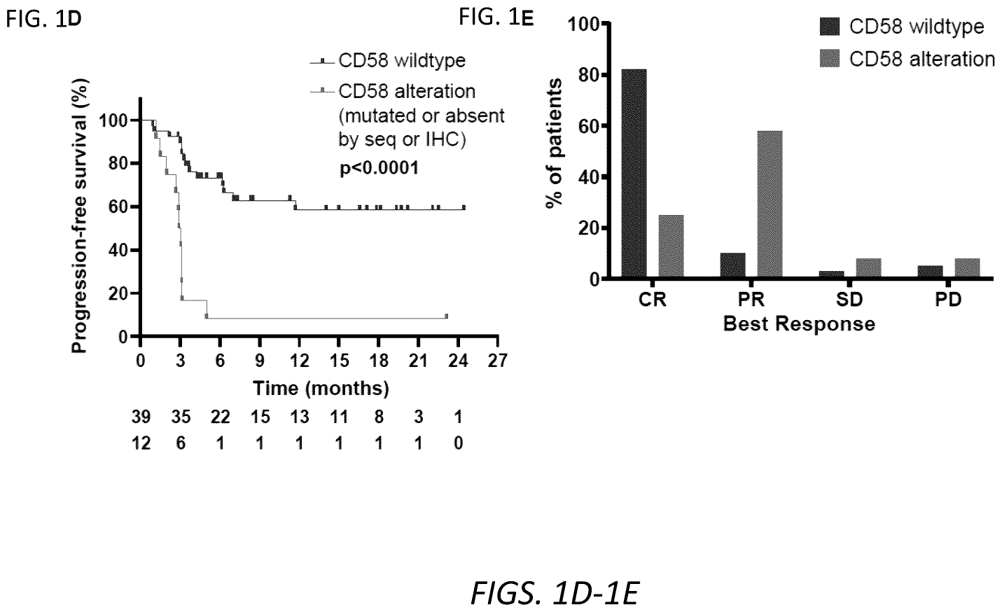

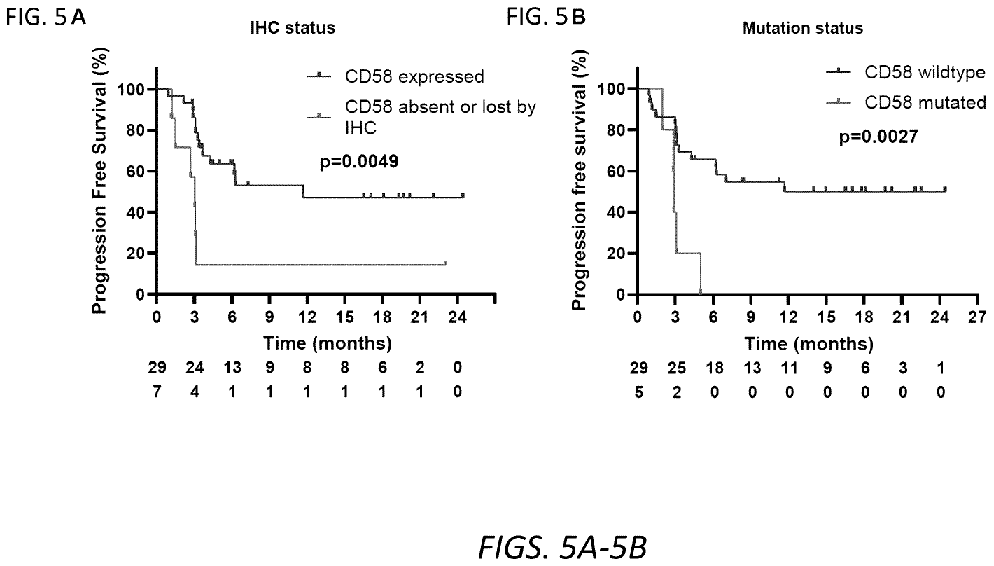

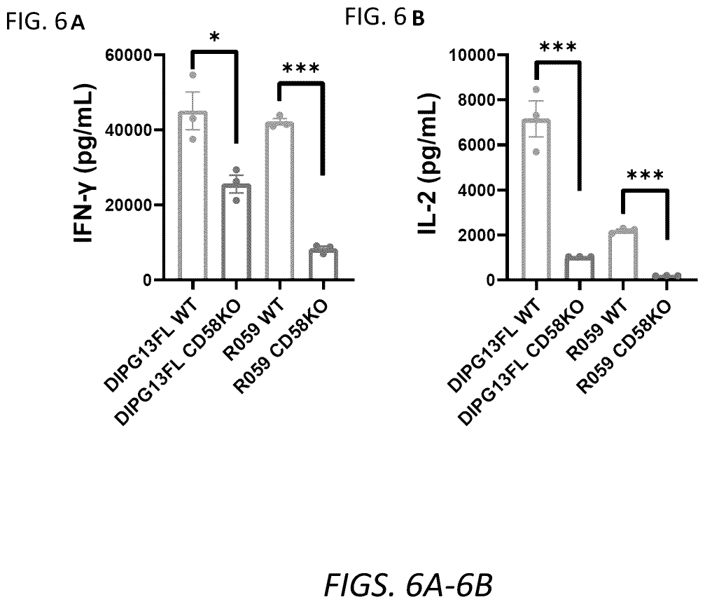

In these studies, seventy (70) LBCL patients were treated with commercially obtained axicabtagene ciloleucel. Response rates in these studies have remained remarkably consistent with the data from other research groups as well as data from the initial clinical trials leading to the drug's approval. To investigate whether CD58 patient status could affect patient outcome, experiments were performed to evaluate CD58 expression on archival tumor tissue for any available tumor samples from patients that received axicabtagene ciloleucel (N=36, including 31 pre-therapy only, 2 post-therapy/progression only, 3 pre- and post-therapy/progression). Tumor microarrays consisting of pre- and post-CAR tumor tissues were built and subsequently stained for CD58 expression by immunohistochemistry (IHC) technique (see, e.g., FIG. 1A). It was observed that patients exhibiting loss of CD58 protein expression (n=7) almost all relapsed within 3.5 months of CAR infusion (see, e.g., FIG. 5A). The only exception was a patient with low disease burden (e.g., one single site of disease) who received bridging radiotherapy prior to lymphodepletion and CAR infusion. It was also observed in this study a case of emergent CD58 loss in response to axicabtagene ciloleucel, with one patient demonstrating loss of CD58 expression only on his post-therapy biopsy (See, e.g., FIG. 1B).

Because CD58 mutations have also been described previously in patients with LBCL, an assessment of CD58 mutation status in patients from the same series for whom pre- and post-treatment plasma were available was performed by using Cancer Personalized Profiling by deep Sequencing (CAPP-seq) in circulating tumor DNA (ctDNA). Out of 34 patients, five patients with CD58 alterations (two single nucleotide variants, K60E and C187R; two frameshift deletions; and one fusion) were identified, all of whom ultimately failed to achieve a durable remission. One of the identified mutations (C187R) was emergent only at disease progression. Both of the identified single nucleotide variants had previously predicted to affect protein folding and function. According to a published crystal structure of the CD58 interaction with CD2, which is its ligand on T cells, the K60E mutation specifically is believed to abrogate the salt bridge through which they interact (See, e.g., FIG. 1C).

Additional studies were performed to evaluate the rates of progression free survival (PFS) post-CAR for patients with and without CD58 aberrations (e.g., loss of protein expression by IHC or mutation(s) found by CAPP-seq) (see, e.g., Table 1). In these studies, it was observed that patients harboring a CD58 aberration had significantly shorter PFS than those without such an aberration (median PFS for CD58 aberration 3 months vs. not reached for CD58 intact, p<0.0001. See, e.g., FIG. 1D). This difference was maintained when evaluating only those patients with available IHC data (median PFS for CD58 non-expressors 3 months vs. 11.7 months for CD58 expressors, p=0.0049. See, e.g., FIG. 5A) or CAPP-seq data (median PFS for CD58 mutations 3 months vs. not reached for CD58 wild-type, p=0.0027. See, e.g., FIG. 5B). In addition, it was observed that patients with a CD58 alteration were also significantly less likely to achieve a complete response (25% vs 82%, p=0.0005) and more likely to achieve a partial response (58% vs. 10%, p=0.0015) (see, e.g., FIG. 1E). Therefore, it was concluded that alterations in expression of wild-type CD58 are highly correlated with durable response in patients with LBCL treated with axicabtagene ciloleucel (YESCARTA®).

TABLE 1
Patient characteristics and outcomes by CD58 status.

| | |
|---|---|
| CD58 WNL | CD58 Aberration |
| (n = 39) | (n = 12) |

| | | |
|---|---|---|
| Subtype | | |
| DLBCL | (n = 27) 69% | (n = 7) 58% |
| TFL | (n = 11) 28% | (n = 3) 25% |
| PBMCL | (n = 1) 3% | (n = 2) 17% |

Age

| | | | | |
|---|---|---|---|---|
| Median, range | 61 | (30-76) | 51 | (21-66) |

| | | |
|---|---|---|
| >65 | (n = 15) 38% | (n = 1) 8% |
| Male % | (n = 25) 64% | (n = 6) 50% |
| Stage III/IV % | (n = 29) 75% | (n = 11) 92% |
| Elevated LDH % | (n = 15) 38% | (n = 9) 75% |
| IPI =>3% | (n = 16) 41% | (n = 7) 58% |
| Bulky % | (n = 4) 10% | (n = 5) 42% |
| Double Hit %* | (n = 7) 20% | (n = 3) 27% |

Lines of therapy

| | | | | |
|---|---|---|---|---|
| Median (range) | 3 | (2-7) | 3 | (2-6) |

| | | |
|---|---|---|
| 3 or more | (n = 21) 54% | (n = 7) 58% |
| Auto, yes % | (n = 11) 28% | (n = 1) 8% |

Best Response

| | | |
|---|---|---|
| CR | (n = 32) 82% | (n = 3) 25% |
| PR | (n = 4) 10% | (n = 7) 58% |
| SD | (n = 1) 3% | (n = 1) 8% |
| PD | (n = 2) 5% | (n = 1) 8% |

(CR: complete response; PR: partial response; SD: stable response; PD: progressive disease; IPI: international prognostic index; WNL: within normal limits; DLBCL: diffuse large B-Cell lymphoma; TFL: transformed follicular lymphoma; and PBMCL: primary mediastinal B-cell lymphoma.

### Example 2

### CD58 Loss Diminishes the Efficacy of CAR-T Cells In Vitro and in Xenograft Models

This Example describes the results of studies performed to illustrate that a loss in CD58 expression diminishes the efficacy of CAR-T cells in vitro and in xenograft models.

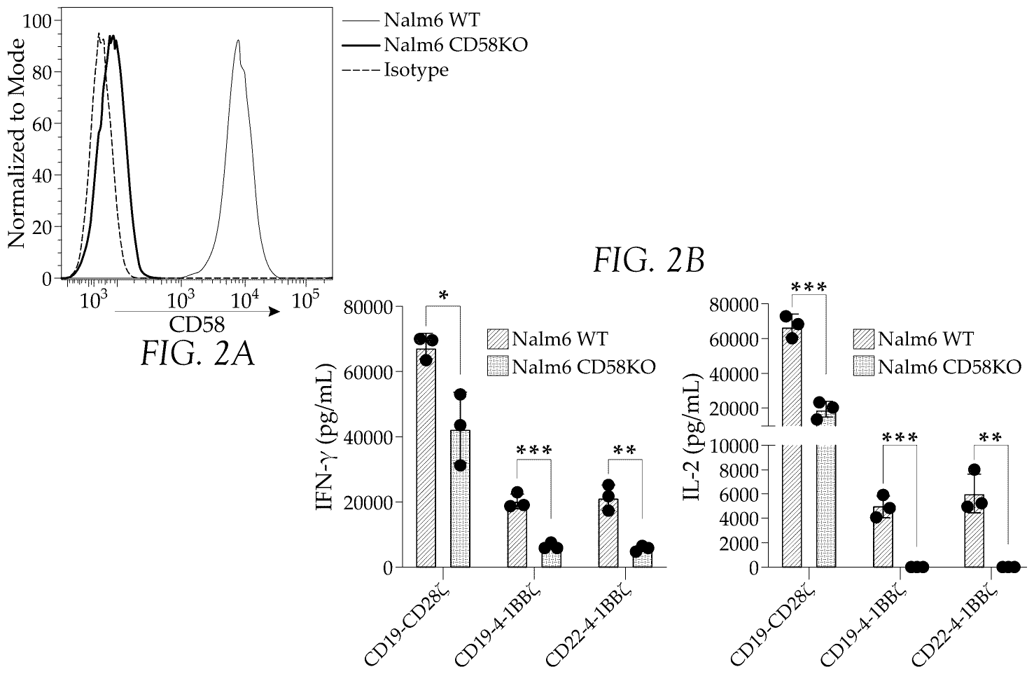

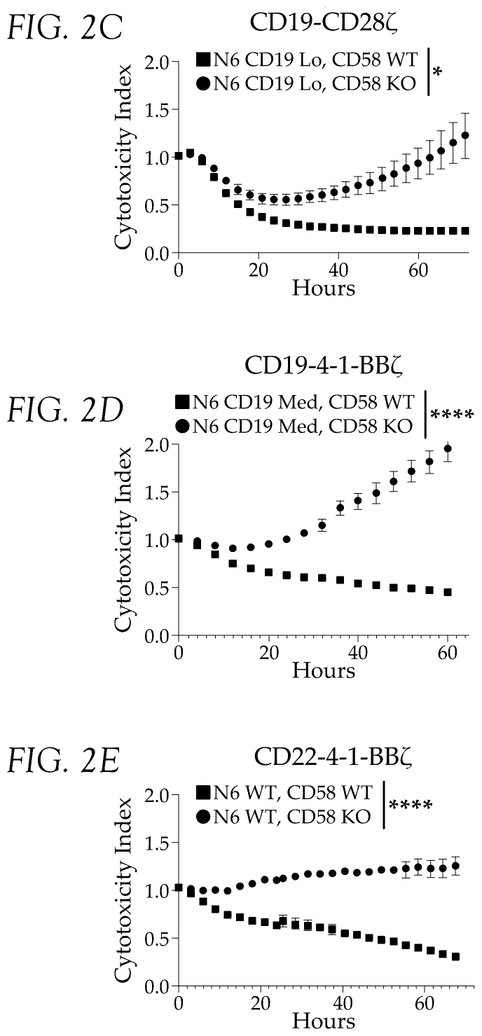

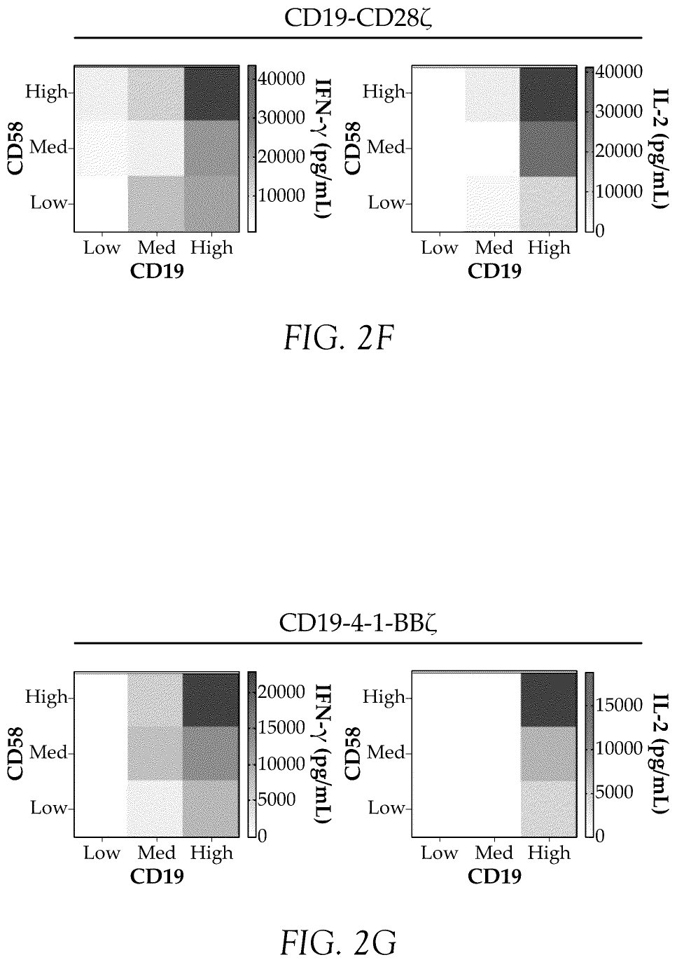

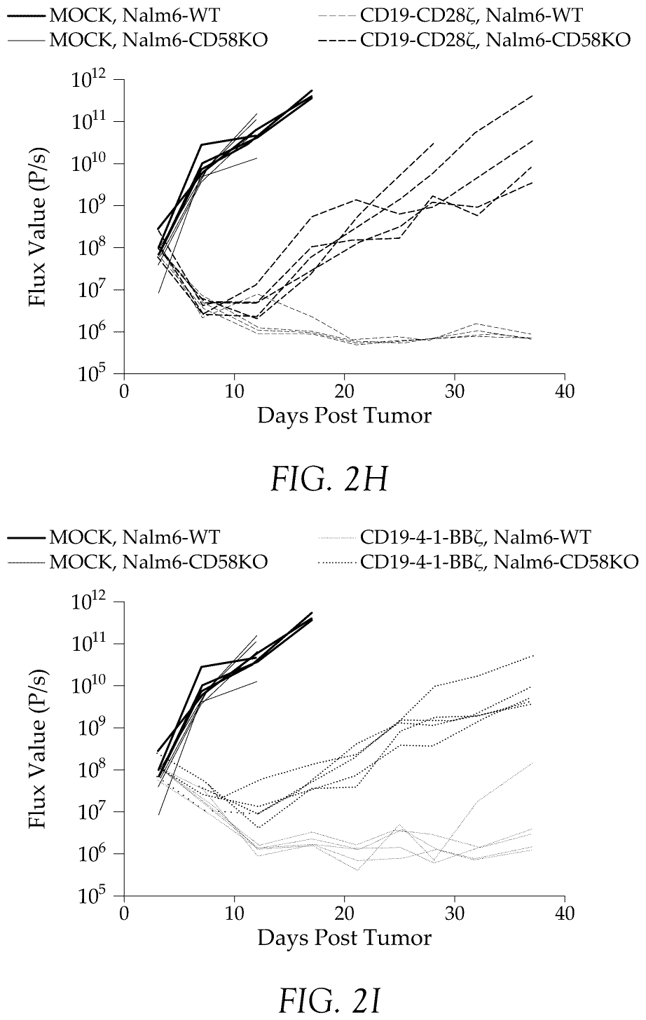

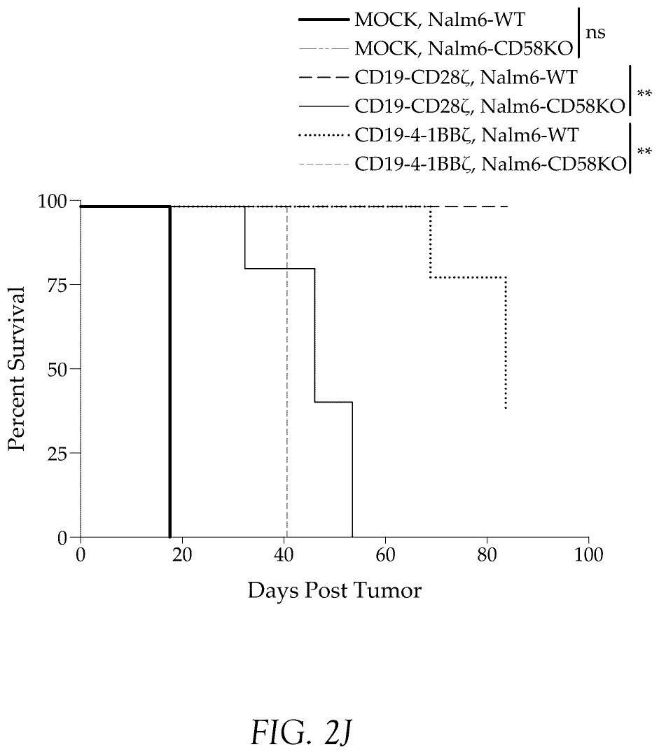

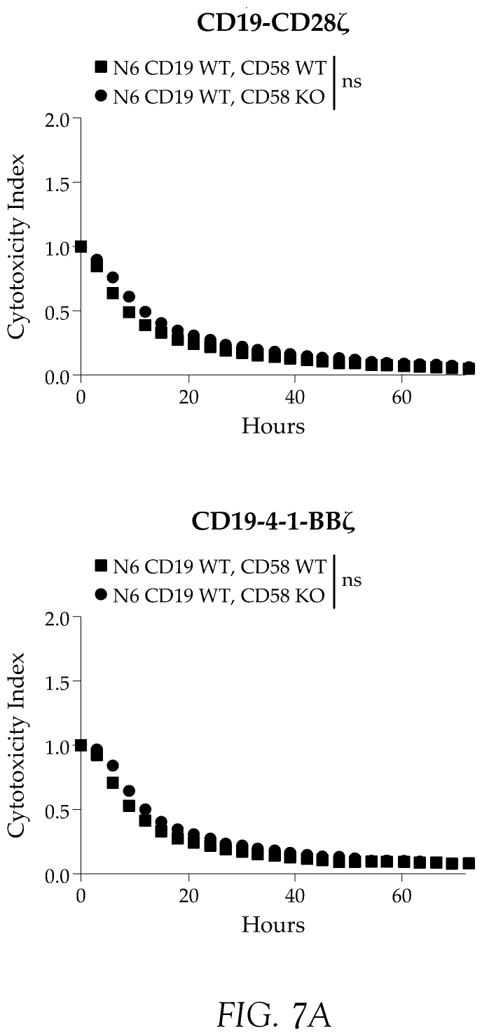

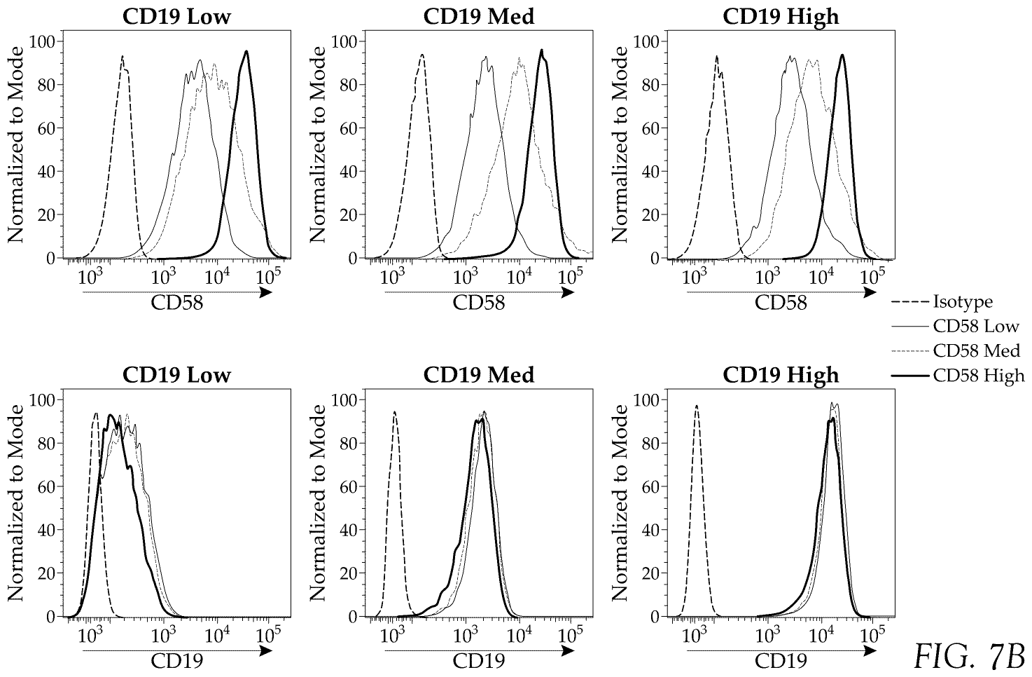

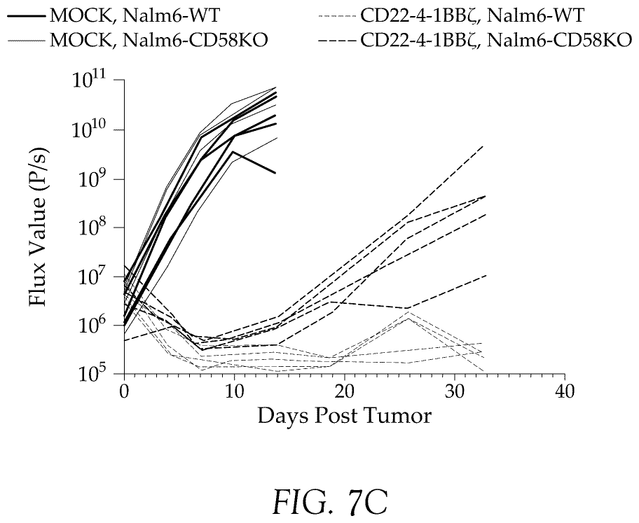

In order to explore the role of CD58 expression on CAR-T cell efficacy, a CRISPR-Cas9 approach was used to knockout CD58 expression from the well-described Nalm6 cell line (FIG. 2A). In these studies, it was observed that both the CD19 CARs contained in axicabtagene ciloleucel (CD19-CD28ζ), tisagenlecleucel (CD19-4-1BBζ) as well as the m971-based CD22 CAR (CD22-4-1BBζ) generated significantly reduced IL-2 and IFN-γ in response to CD58 knockout tumor cells vs wild-type tumor cells (see, e.g., FIG. 2C). Note that m971 is a membrane proximal binding anti CD22 scFv. The same reduction in cytokine production by a GD2-4-1BBζ CAR was also observed when incubated with DIPG cell lines with and without CD58 knockout (see, e.g., FIG. 7A).

Subsequently, Incucyte assays that measured tumor death over several days were carried out in order to assess cytotoxicity. When CD19 density was adequate, as on the wild-type Nalm6 line, no differences in killing were observed for either CD19-CD28ζ CAR or CD19-4-1BBζ CAR against CD58-wild-type or CD58-knockout lines (see, e.g., FIG. 7A). However, when CAR-T cells were tested against cell lines with reduced CD19 expression, significant differences emerged, with CD19 CAR T cells demonstrated reduced cytotoxicity against CD58-knockout vs wild-type cell lines (see, e.g., FIGS. 2C-2D). As CD22 density on the Nalm6 cell line is lower, differences in killing of CD58-knockout vs wild-type cells by CD22-4-1BBζ CAR were observed in the native antigen density range (see, e.g., FIG. 2E).

To further explore the interaction of CD19 antigen density and CD58 expression on CAR function, Nalm6 clones expressing varying levels of both CD19 and CD58 were generated through knockout, overexpression, FACS sorting, and single cell cloning (see, e.g., FIG. 7B). It was observed that cytokine production by CAR-T cells in response to tumor was dependent on both the target antigen density (for instance, CD19) and CD58 density on tumor cells (FIGS. 2F-2G). Only when both were sufficiently high, as they are on the Nalm6 wild-type line, is maximal cytokine production achieved.

Additional studies were carried out to evaluate CAR efficacy in vivo against CD58-knockout xenografts and wild-type xenografts. In these studies, while both CD19-CD28ζ and CD19-4-1BBζ CARs demonstrated initial efficacy in mice bearing CD58-knockout Nalm6, they were unable to clear disease, resulting in eventual tumor outgrowth (see, e.g., FIGS. 2H-2I), reflecting the experience with human patients in which most patients achieved a partial response (PR) before eventual progression. A similar trend was observed with the CD22-4-1BBζ CAR (see, e.g., FIG. 7C). While both CD19 CARs resulted in long term cures in mice bearing CD58-wild-type xenografts, neither was able to cure mice with CD58-knockout tumors (see, e.g., FIG. 2J).

### Example 3

### CD58-CD2 Interactions are Responsible for Enhanced CAR-T Cell Activity

This Example describes the results of experiments performed to demonstrate that CD58 interaction with CD2 are responsible for enhanced CAR-T cell activity.

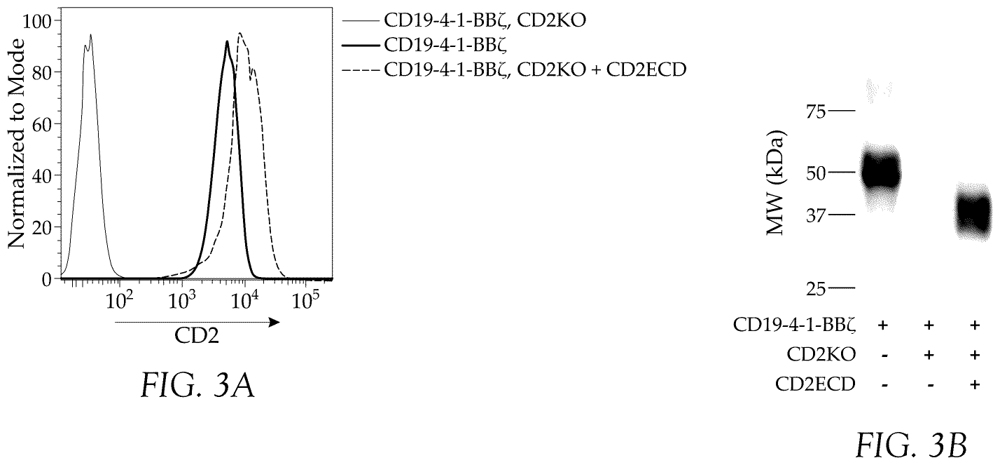

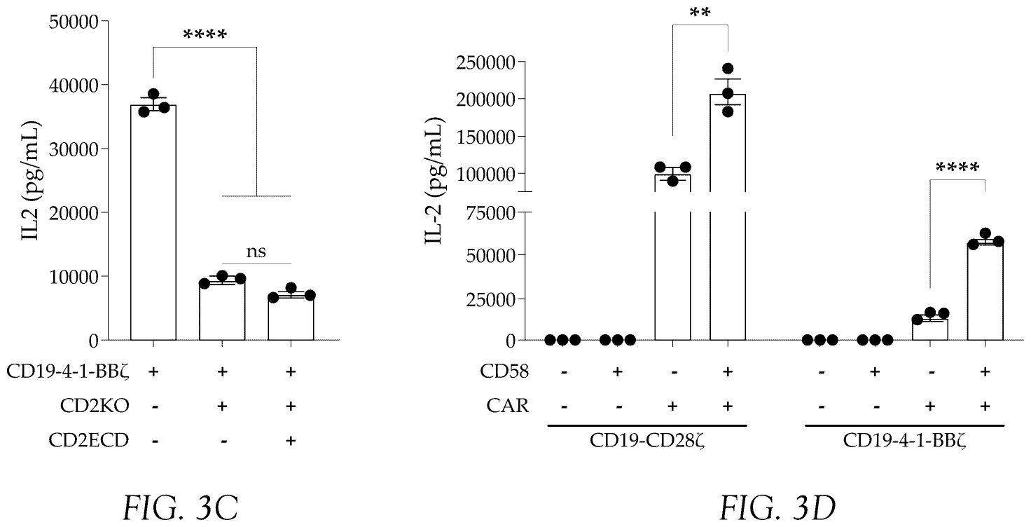

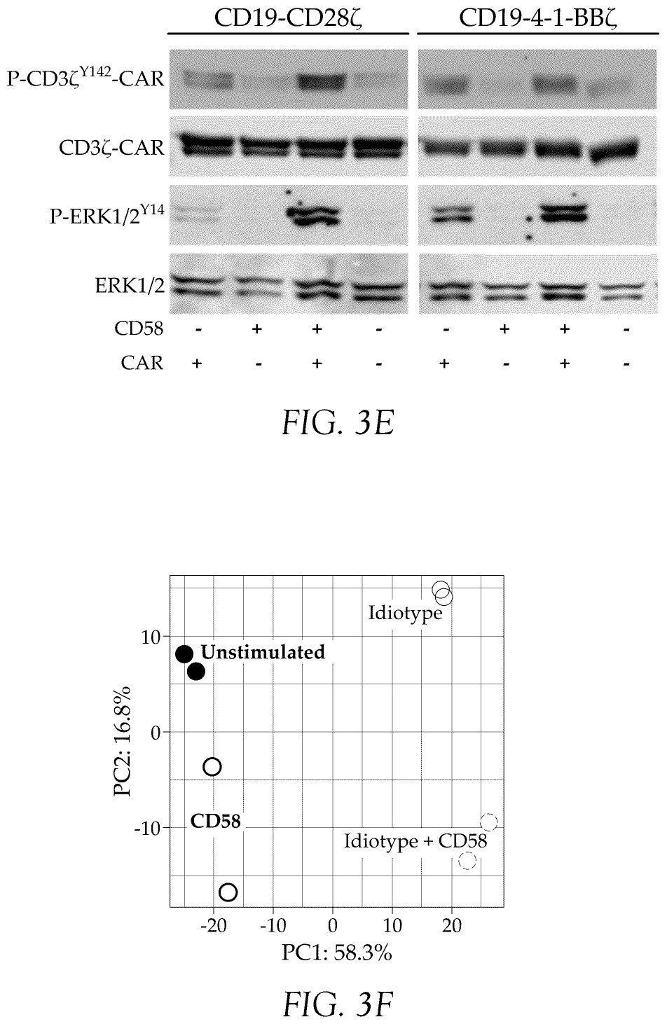

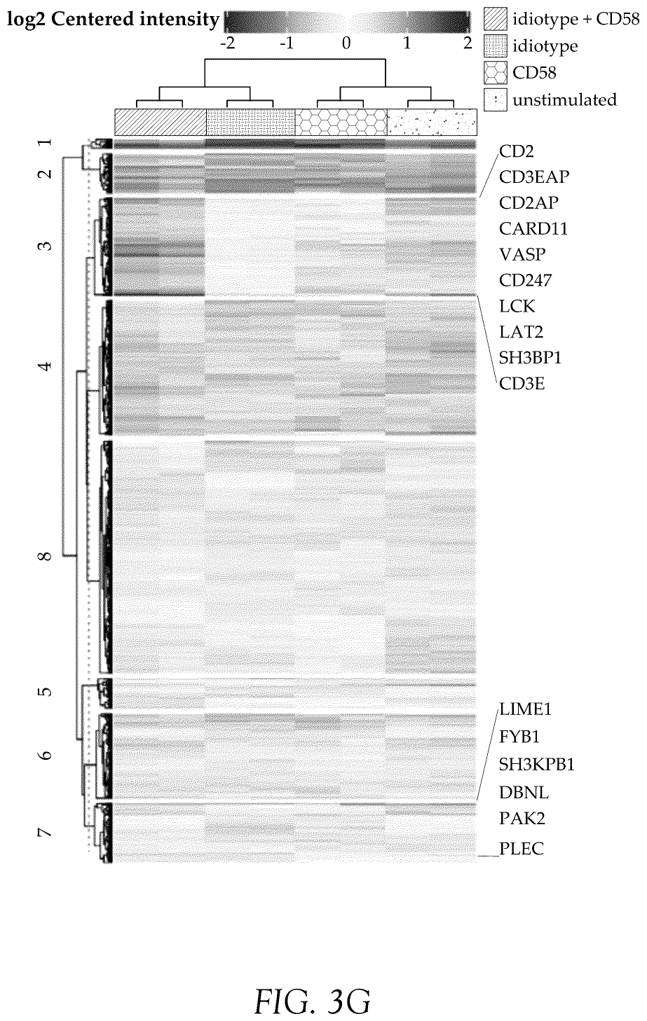

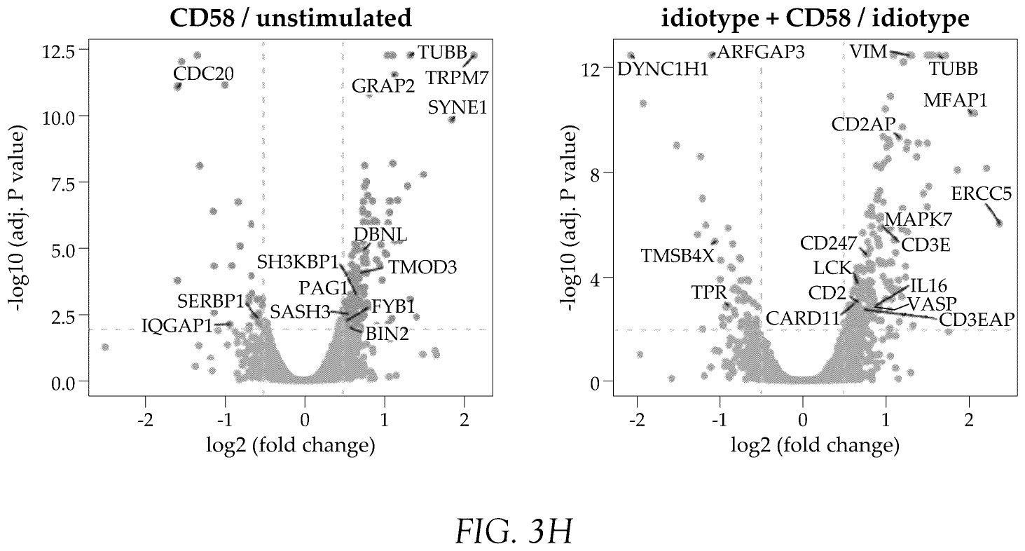

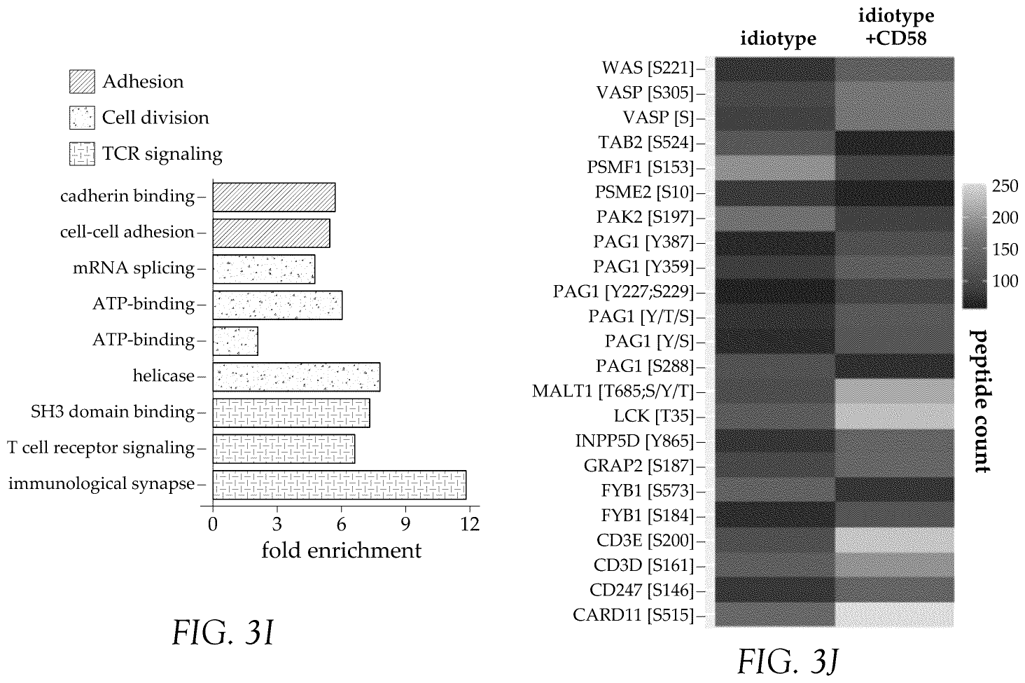

As described above, the natural ligand for CD58 is CD2, a costimulatory molecule highly expressed by most T cells. To test the importance of CD2 expression and signaling, a T cell line (Jurkat) expressing a CD19 CAR was generated, in which CD2 was knocked-out (CD2KO, FIGS. 3A-3B). It was observed that CD2KO CAR cells generated significantly less IL-2 in response to antigen encounter than CD2 WT CAR cells (FIG. 3C). To test the contribution of the intracellular signaling domain of CD2 to CAR efficacy, a variant of CD2 expressing only the extracellular domain of CD2 (CD2-ECD) was re-expressed in these cells (FIGS. 3A-3B) and found that this CD2 variant did not rescue CAR function (see, e.g., FIG. 3C). Therefore, signaling by the CD2 intracellular domain can be required for efficient CAR-T cell function.

Additional studies were performed to explore the contribution of CD2 ligation on CAR-T cell downstream signaling. In these studies, it was found that crosslinking CD19 CAR-T cells with idiotype antibody, CD58 protein, or idiotype+CD58, significantly enhanced TL-2 production by both CD19-CD28ζ and CD19-4-1BBζ CAR-T cells when both CAR and CD2 were ligated (FIG. 3D). Additionally, after a five-minute stimulation, it was observed significantly higher phosphorylation of both CD3ζ-CAR and downstream ERK in CAR-T cells activated with both idiotype antibody and CD58 (FIG. 3E). This observation is in agreement with previous findings demonstrating that CD2 enhances signaling by the proximal T cell receptor machinery.

To more fully explore the role of CD2 signaling in CAR-T cells, protein lysates from the experiment described above were taken and measured 15,993 phosphopeptides mapping to 3372 proteins using mass spectrometry. It was found that principle component analysis of the phosphoproteome showed biological replicates clustered together, demonstrating reproducibility between donors. In these studies, principle component 1 (PC1) captured 58% of the variance, separating samples based on treatment with anti-idiotype and indicating the largest differences were due to stimulation through the CAR. Principle component 2 (PC2) accounted for 17% of the variance and separated samples based on treatment with soluble CD58 (FIG. 3F).

As showed in FIG. 3G, unsupervised clustering of differentially abundant peptides showed that the majority of peptides (clusters 1, 2, 4, and 8) were elevated in both idiotype treated conditions and therefore can be attributed to signaling though the CAR. Phosphopeptides in cluster 7 were elevated exclusively in conditions treated with CD58, indicating that CD2 signaling is responsible for these changes. Notably, cluster 7 contained several SH3 domain peptides including SH3KBP1 and DBNL, which are described as modulators of the actin cytoskeleton and known to play a role in T cell polarization. Cluster 3 contained phosphopeptides that were most abundant in the condition stimulated with both idiotype and CD58 and included many core components of the TCR signaling pathway such as LCK, CD3ε, and CD247 (CD3ζ). These results show that CD2 co-stimulation synergizes with CAR signaling to increase activation through common TCR pathways. Additionally, cluster 3 included CD2 and its signaling adapter protein CD2AP, indicating that signaling through the CAR augments activation of the CD2 pathway. CD2 co-stimulation alone resulted in differential abundance of 157 phosphopeptides, while CD2 co-stimulation in addition to CAR activation resulted in 236 differentially abundant peptides (FIG. 3H), further supporting the notion that the effect of signaling through these two pathways is distinct.

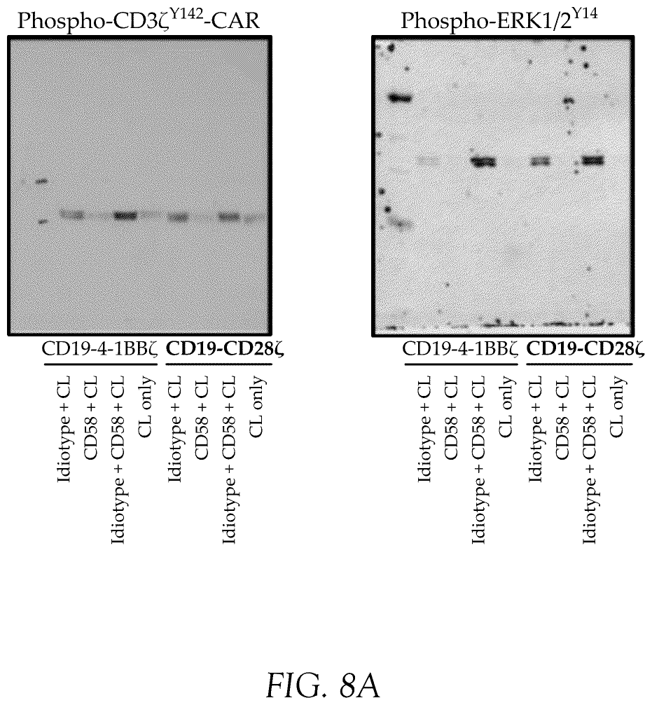

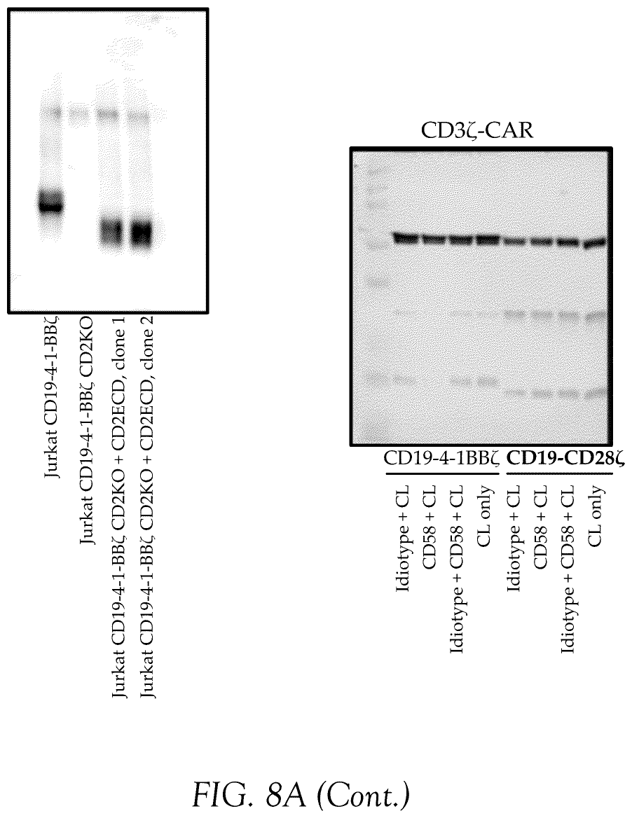

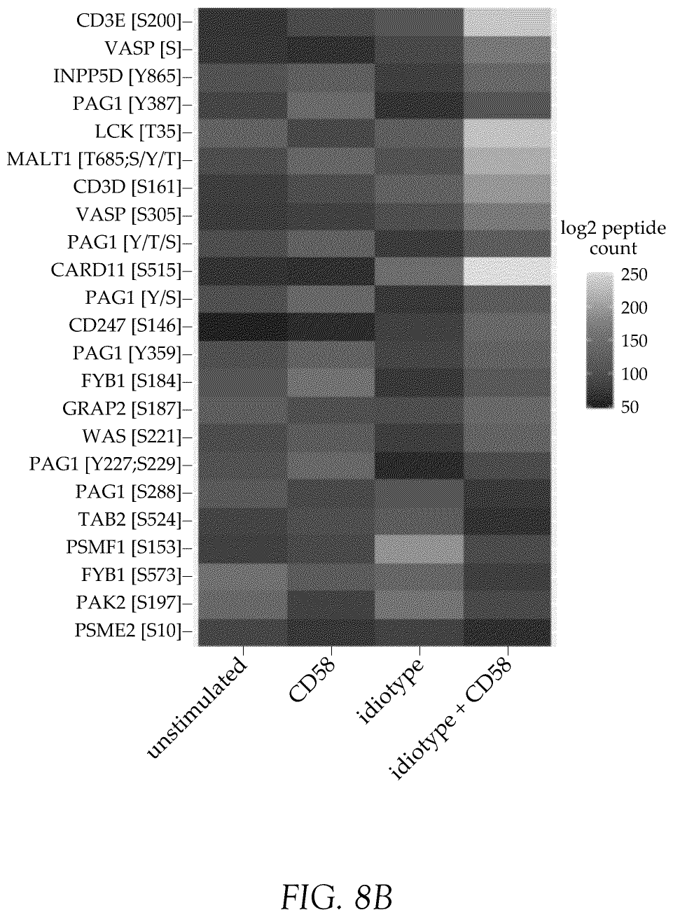

Bioinformatics analysis of phosphopeptides regulated by CD2 stimulation in the presence of CAR signaling revealed significant enrichment of Gene Ontology (GO) terms including “cell-cell adhesion”, and “immunological synapse” (FIG. 3I). These results are in agreement with previous reports that CD2 may be essential for cytoskeletal polarization towards the target cell. To further examine the role of CD2 in potentiating TCR signaling, additional studies were performed to examine phosphopeptides corresponding to genes in the Reactome TCR Signaling gene set. It was found that CD2 ligation by CD58 resulted in enhanced TCR pathway signaling with increases in phosphorylated LCK, CD3δ, CD3ε, and GRAP2. Additionally, proteins involved in actin cytoskeleton reorganization such as vasodilator-stimulated phosphoprotein (VASP) and Wiskott-Aldrich syndrome (WAS) protein were elevated in CD2 stimulated cells (FIG. 3J and FIG. 8). Notably, phosphopeptides corresponding to MALT1 and CARD 11, key components of the CARD 11-BCL10-MALT1 (CBM) signalosome complex, were also increased with CD2 ligation. The CBM signalosome may be an important molecular link between T cell surface signaling and NF-kB activation, which may be required for T cell proliferation, survival, and effector function.

### Example 4

### Car-T Cells can be Engineered to Overcome CD58 Loss in B Cell Malignancies

This Example describes the results of experiments performed to illustrate that CAR-T cells can be engineered to overcome CD58 loss in B cell malignancies.

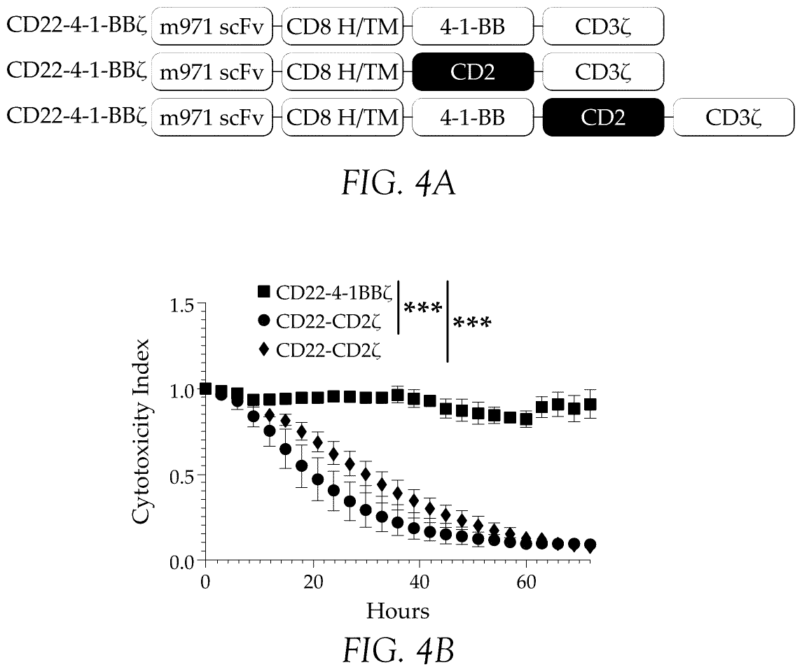

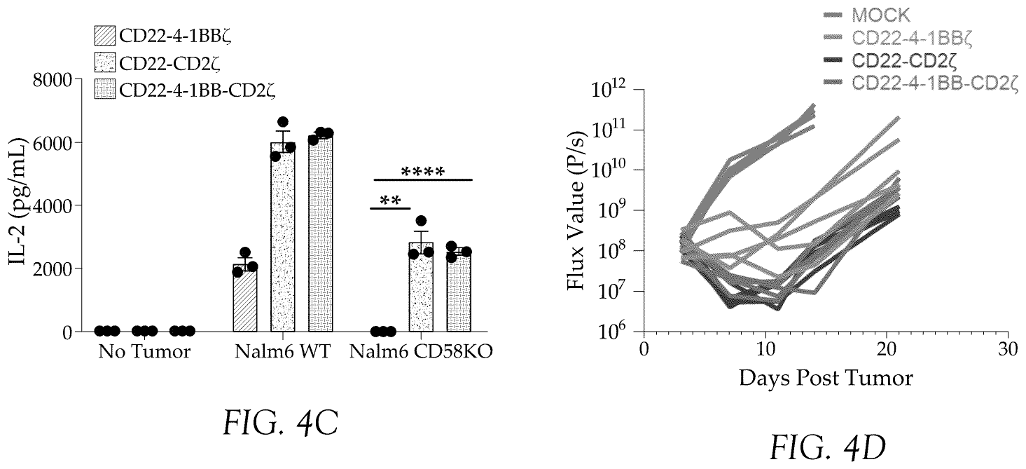

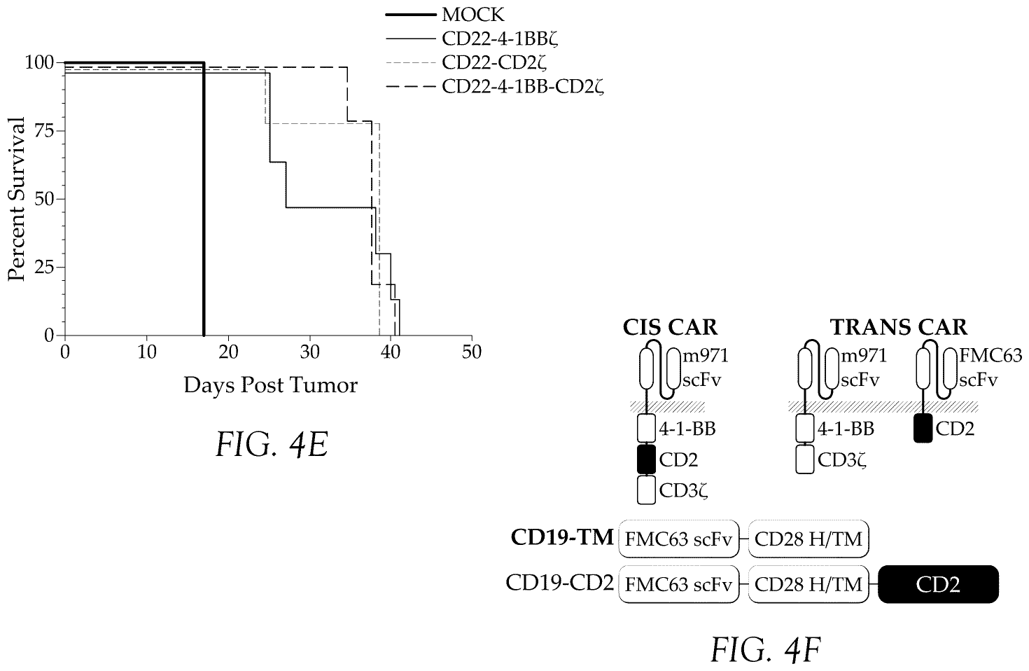

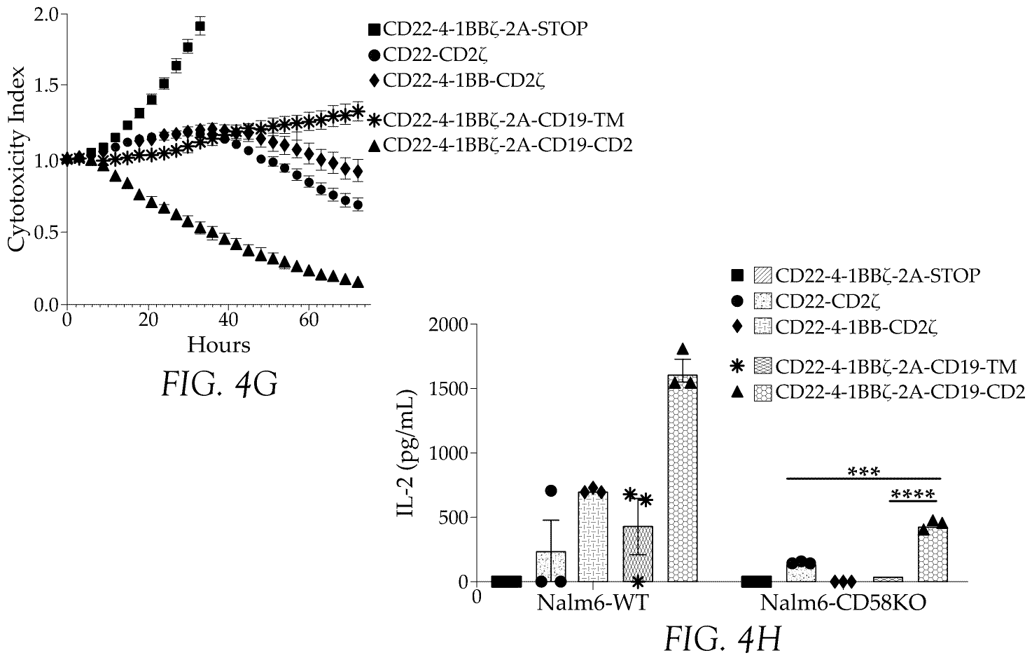

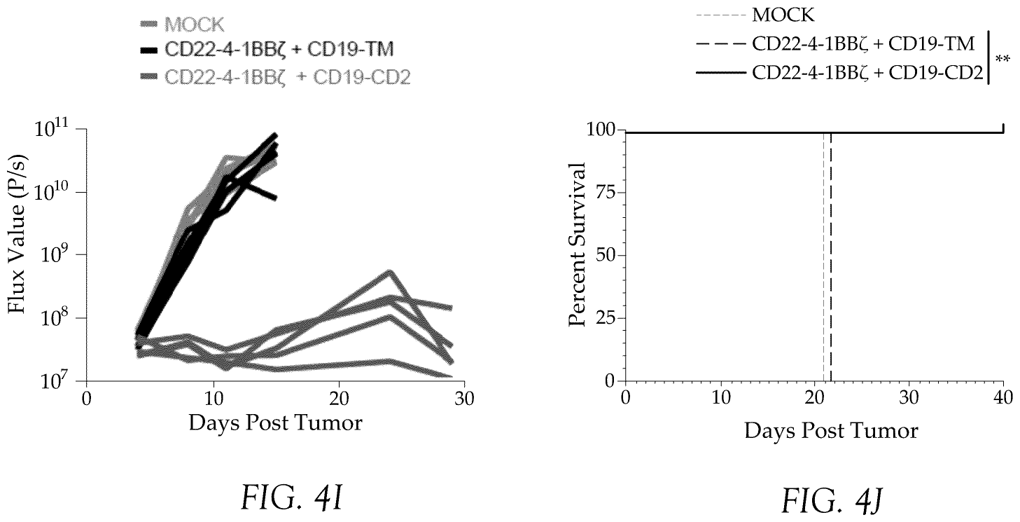

As CD2 signaling due to CD58 ligation enhances CAR-T cell function, second and third generation CAR-T cells were generated by integrating CD2 costimulatory domains into the CAR molecule. See, CD22-CD2ζ and CD22-4-1BB-CD2ζ in FIG. 4A. The CD22-4-1BBζ CAR was chosen for these experiments because the effects of CD58 knockout were found more apparent in vitro, facilitating more thorough testing of multiple constructs. A comparison was performed for these CAR constructs (CD22-CD2ζ and CD22-4-1BB-CD2ζ) against the CD22-4-1BBζ CAR, where it was found that they were able to kill CD58-knockout Nalm6 cells when the 4-1BBζ CAR could not (FIG. 4B). Additionally, they generated additional TL-2 in response to co-culture with both CD58 wild-type and CD58 knockout Nalm6 lines (FIG. 4C). However, in vivo, while the CD2-containing CARs initially enhanced tumor control of CD58 knockout xenografts (FIG. 4D), tumor eventually grew out in all mice, abrogating any benefit in survival (FIG. 4E).

In a native T cell, CD2 signaling occurs in trans to the T cell receptor. To better mimic the relationship of CD2 to the TCR, a trans CAR construct was generated in which the anti-CD19 single chain variable fragment (scFv) FMC63 was fused to a transmembrane domain and the CD2 intracellular domain (FIG. 4F). In these experiments, the two constructs can be expressed in one cell using two viral vectors or in a single bicistronic vector. In some experiments where bicistronic vectors were used, a connector sequence was inserted between the sequences for the two CARs. Below is an exemplary connector sequence used in these experiments:

(SEQ ID NO: 11)
RKRREFATNFSLLKQAGDVEENPGPLE.

The coding sequence for the above connector sequence was as follows:

(SEQ ID NO: 12)
CGCAAGAGAAGAGAATTCgcaacaaacttctctct
gctgaaacaagccggagatgtcgaagagaatcctg
gaccgCTCGAG

wherein: RKRR (SEQ ID NO: 13) corresponds to a furin cleavage sequence added downstream of the first CAR sequence.
EF corresponds to an EcoRI cut site;
ATNFSLLKQAGDVEENPGP (SEQ ID NO: 14) corresponds to an auproteolytic peptide sequence from porcine teschovirus-1 2A (P2A).
LE corresponds to an XhoI cut site.

In cytotoxicity assays, T cells expressing both the CD22-4-1BBζ and a CD19-CD2 receptor in trans were able to kill CD58-knockout tumor cells, whereas the second and third generation CARs failed (FIG. 4G). The same trend was observed for cytokine production, with the trans configuration generating the most TL-2 against the CD58-knockout line (FIG. 411). In particular, control T cells expressing the CD22-4-1BBζ CAR and a CD19 scFv-transmembrane domain without intracellular CD2 signaling domain did not recover the ability to kill or generate cytokine against CD58 knockout cells, indicating that CD2 signaling may be essential to the CAR/tumor cell interaction (FIGS. 4G-4H). Indeed, in vivo, T cells expressing CD22-4-1BBζ and a CD19-CD2 receptor in trans effectively controlled CD58-knockout tumor growth compared to the control construct (FIG. 4I), resulting in significantly improved survival (FIG. 4J). Therefore, it was concluded that next generation CARs for lymphoma that integrate CD2 signaling in a trans approach are effective at overcoming CD58 loss, which is a common but novel mechanism of CAR resistance uncovered by the studies described herein.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### REFERENCES

1 Majzner, R. G. & Mackall, C. L. Clinical lessons learned from the first leg of the CAR T cell journey. Nat Med 25, 1341-1355, doi:10.1038/s41591-019-0564-6 (2019).
2 Neelapu, S. S. et al. Axicabtagene Ciloleucel CAR T-Cell Therapy in Refractory Large B-Cell Lymphoma. N Engl J Med 377, 2531-2544, doi:10.1056/NEJMoa1707447 (2017).
3 Schuster, S. J. et al. Tisagenlecleucel in Adult Relapsed or Refractory Diffuse Large B-Cell Lymphoma. N Engl J Med 380, 45-56, doi:10.1056/NEJMoal804980 (2019).
4 Locke, F. L. et al. Long-term safety and activity of axicabtagene ciloleucel in refractory large B-cell lymphoma (ZUMA-1): a single-arm, multicentre, phase 1-2 trial. Lancet Oncol 20, 31-42, doi:10.1016/S1470-2045(18)30864-7 (2019).
5 Nastoupil, L. J. et al. Standard-of-Care Axicabtagene Ciloleucel for Relapsed or Refractory Large B-Cell Lymphoma: Results From the US Lymphoma CAR T Consortium. J Clin Oncol, JC01902104, doi:10.1200/JCO.19.02104 (2020).
6 Brudno, J. N. & Kochenderfer, J. N. Chimeric antigen receptor T-cell therapies for lymphoma. Nat Rev Clin Oncol 15, 31-46, doi:10.1038/nrclinonc.2017.128 (2018).
7 June, C. H. & Sadelain, M. Chimeric Antigen Receptor Therapy. N Engl J Med 379, 64-73, doi:10.1056/NEJMra1706169 (2018).
8 Abramson, J. S. et al. Pivotal Safety and Efficacy Results from Transcend NHL 001, a Multicenter Phase 1 Study of Lisocabtagene Maraleucel (liso-cel) in Relapsed/Refractory (R/R) Large B Cell Lymphomas. Blood 134, 241-241, doi:10.1182/blood-2019-127508 (2019).
9 Chow, V. A., Shadman, M. & Gopal, A. K. Translating anti-CD19 CAR T-cell therapy into clinical practice for relapsed/refractory diffuse large B-cell lymphoma. Blood 132, 777-781, doi:10.1182/blood-2018-04-839217 (2018).
10 Brudno, J. N. et al. Safety and feasibility of anti-CD19 CAR T cells with fully human binding domains in patients with B-cell lymphoma. Nat Med 26, 270-280, doi:10.1038/s41591-019-0737-3 (2020).
11 Shah, N. N. & Fry, T. J. Mechanisms of resistance to CAR T cell therapy. Nat Rev Clin Oncol 16, 372-385, doi:10.1038/s41571-019-0184-6 (2019).
12 Majzner, R. G. & Mackall, C. L. Tumor Antigen Escape from CAR T-cell Therapy. Cancer Discov 8, 1219-1226, doi:10.1158/2159-8290.CD-18-0442 (2018).
13 Shalabi, H. et al. Sequential loss of tumor surface antigens following chimeric antigen receptor T-cell therapies in diffuse large B-cell lymphoma. Haematologica, doi:10.3324/haematol.2017.183459 (2018).
14 Fry, T. J. et al. CD22-targeted CAR T cells induce remission in B-ALL that is naive or resistant to CD19-targeted CAR immunotherapy. Nat Med 24, 20-28, doi:10.1038/nm.4441 (2018).
15 Neelapu, S. S. et al. CD19-Loss with Preservation of Other B Cell Lineage Features in Patients with Large B Cell Lymphoma Who Relapsed Post-Axi-Cel. Blood 134, 203-203, doi:10.1182/blood-2019-126218 (2019).
16 Fraietta, J. A. et al. Determinants of response and resistance to CD19 chimeric antigen receptor (CAR) T cell therapy of chronic lymphocytic leukemia. Nat Med 24, 563-571, doi:10.1038/s41591-018-0010-1 (2018).
17 Finney, O. C. et al. CD19 CAR T cell product and disease attributes predict leukemia remission durability. J Clin Invest 129, 2123-2132, doi:10.1172/JCI125423 (2019).
18 Maude, S. L. et al. Tisagenlecleucel in Children and Young Adults with B-Cell Lymphoblastic Leukemia. N Engl J Med 378, 439-448, doi:10.1056/NEJMoa1709866 (2018).
19 Majzner, R. G. et al. Tuning the Antigen Density Requirement for CAR T-cell Activity. Cancer Discov 10, 702-723, doi:10.1158/2159-8290.CD-19-0945 (2020).
20 Gardner, R. A. et al. Intent-to-treat leukemia remission by CD19 CAR T cells of defined formulation and dose in children and young adults. Blood 129, 3322-3331, doi:10.1182/blood-2017-02-769208 (2017).
21 Lee, D. W. et al. T cells expressing CD19 chimeric antigen receptors for acute lymphoblastic leukaemia in children and young adults: a phase 1 dose-escalation trial. Lancet 385, 517-528, doi:10.1016/S0140-6736(14)61403-3 (2015).
22 Turtle, C. J. et al. CD19 CAR-T cells of defined CD4+:CD8+ composition in adult B cell ALL patients. J Clin Invest 126, 2123-2138, doi:10.1172/JCI85309 (2016).
23 Veltroni, M. et al. Expression of CD58 in normal, regenerating and leukemic bone marrow B cells: implications for the detection of minimal residual disease in acute lymphocytic leukemia. Haematologica 88, 1245-1252 (2003).
24 Chen, J. S. et al. Identification of novel markers for monitoring minimal residual disease in acute lymphoblastic leukemia. Blood 97, 2115-2120, doi:10.1182/blood.v97.7.2115 (2001).
25 Jacob, M. C. et al. Quantification of cellular adhesion molecules on malignant B cells from non-Hodgkin's lymphoma. Leukemia 13, 1428-1433, doi:10.1038/sj.leu.2401517 (1999).
26 Cao, Y. et al. Mutations or copy number losses of CD58 and TP53 genes in diffuse large B cell lymphoma are independent unfavorable prognostic factors. Oncotarget 7, 83294-83307, doi:10.18632/oncotarget.13065 (2016).
27 Challa-Malladi, M. et al. Combined genetic inactivation of beta2-Microglobulin and CD58 reveals frequent escape from immune recognition in diffuse large B cell lymphoma. Cancer Cell 20, 728-740, doi:10.1016/j.ccr.2011.11.006 (2011).
28 Newman, A. M. et al. An ultrasensitive method for quantitating circulating tumor DNA with broad patient coverage. Nat Med 20, 548-554, doi:10.1038/nm.3519 (2014).
29 Adzhubei, I. A. et al. A method and server for predicting damaging missense mutations. Nat Methods 7, 248-249, doi:10.1038/nmeth0410-248 (2010).
30 Wang, J. H. et al. Structure of a heterophilic adhesion complex between the human CD2 and CD58 (LFA-3) counterreceptors. Cell 97, 791-803, doi:10.1016/s0092-8674(00)80790-4 (1999).
31 Barrett, D. M. et al. Noninvasive bioluminescent imaging of primary patient acute lymphoblastic leukemia: a strategy for preclinical modeling. Blood 118, el 12-117, doi:10.1182/blood-2011-04-346528 (2011).
32 Haso, W. et al. Anti-CD22-chimeric antigen receptors targeting B-cell precursor acute lymphoblastic leukemia. Blood 121, 1165-1174, doi:10.1182/blood-2012-06-438002 (2013).
33 Mount, C. W. et al. Potent antitumor efficacy of anti-GD2 CAR T cells in H3-K27M(+) diffuse midline gliomas. Nat Med 24, 572-579, doi:10.1038/s41591-018-0006-x (2018).
34 Shaw, S. et al. Two antigen-independent adhesion pathways used by human cytotoxic T-cell clones. Nature 323, 262-264, doi:10.1038/323262a0 (1986).
35 Selvaraj, P. et al. The T lymphocyte glycoprotein CD2 binds the cell surface ligand LFA-3. Nature 326, 400-403, doi:10.1038/326400a0 (1987).
36 Demetriou, P. et al. CD2 expression acts as a quantitative checkpoint for immunological synapse structure and T-cell activation. bioRxiv, 589440, doi:10.1101/589440 (2019).
37 Tibaldi, E. V. & Reinherz, E. L. CD2BP3, CIN85 and the structurally related adaptor protein CMS bind to the same CD2 cytoplasmic segment, but elicit divergent functional activities. Int Immunol 15, 313-329, doi:10.1093/intimm/dxg032 (2003).
38 Le Bras, S. et al. Recruitment of the actin-binding protein HIP-55 to the immunological synapse regulates T cell receptor signaling and endocytosis. J Biol Chem 279, 15550-15560, doi:10.1074/jbc.M312659200 (2004).
39 Dustin, M. L. et al. A novel adaptor protein orchestrates receptor patterning and cytoskeletal polarity in T-cell contacts. Cell 94, 667-677, doi:10.1016/s0092-8674(00)81608-6 (1998).
40 Zurli, V. et al. Phosphoproteomics of CD2 signaling reveals AMPK-dependent regulation of lytic granule polarization in cytotoxic T cells. Sci Signal 13, doi:10.1126/scisignal.aaz1965 (2020).
41 Jassal, B. et al. The reactome pathway knowledgebase. Nucleic Acids Res 48, D498-D503, doi:10.1093/nar/gkz1031 (2020).
42 Thome, M., Charton, J. E., Pelzer, C. & Hailfinger, S. Antigen receptor signaling to NF-kappaB via CARMA1, BCL10, and MALT1. Cold Spring Harb Perspect Biol 2, a003004, doi:10.1101/cshperspect.a003004 (2010).
43 Crump, M. et al. Outcomes in refractory diffuse large B-cell lymphoma: results from the international SCHOLAR-1 study. Blood 130, 1800-1808, doi:10.1182/blood-2017-03-769620 (2017).
44 Neelapu, S. S. et al. A Comparison of Two-Year Outcomes in ZUMA-1 (Axicabtagene Ciloleucel) and SCHOLAR-1 in Patients with Refractory Large B Cell Lymphoma. Blood 134, 4095-4095, doi:10.1182/blood-2019-125792 (2019).
45 Bierer, B. E., Peterson, A., Gorga, J. C., Herrmann, S. H. & Burakoff, S. J. Synergistic T cell activation via the physiological ligands for CD2 and the T cell receptor. J Exp Med 168, 1145-1156, doi:10.1084/jem.168.3.1145 (1988).
46 Hahn, W. C., Rosenstein, Y., Calvo, V., Burakoff, S. J. & Bierer, B. E. A distinct cytoplasmic domain of CD2 regulates ligand avidity and T-cell responsiveness to antigen. Proc Natl Acad Sci USA 89, 7179-7183, doi:10.1073/pnas.89.15.7179 (1992).
47 Cheadle, E. J. et al. Ligation of the CD2 co-stimulatory receptor enhances IL-2 production from first-generation chimeric antigen receptor T cells. Gene Ther 19, 1114-1120, doi:10.1038/gt.2011.192 (2012).
48 Kanner, S. B., Damle, N. K., Blake, J., Aruffo, A. & Ledbetter, J. A. CD2/LFA-3 ligation induces phospholipase-C gamma 1 tyrosine phosphorylation and regulates CD3 signaling. J Immunol 148, 2023-2029 (1992).
49 Espagnolle, N. et al. CD2 and TCR synergize for the activation of phospholipase Cgamma1/calcium pathway at the immunological synapse. Int Immunol 19, 239-248, doi:10.1093/intimm/dx1141 (2007).
50 Hubert, P., Debre, P., Boumsell, L. & Bismuth, G. Tyrosine phosphorylation and association with phospholipase C gamma-1 of the GAP-associated 62-kD protein after CD2 stimulation of Jurkat T cell. J Exp Med 178, 1587-1596, doi:10.1084/jem.178.5.1587 (1993).
51 Yang, H. & Reinherz, E. L. Dynamic recruitment of human CD2 into lipid rafts. Linkage to T cell signal transduction. J Biol Chem 276, 18775-18785, doi:10.1074/jbc.M009852200 (2001).
52 Li, J., Smolyar, A., Sunder-Plassmann, R. & Reinherz, E. L. Ligand-induced conformational change within the CD2 ectodomain accompanies receptor clustering: implication for molecular lattice formation. J Mol Biol 263, 209-226, doi:10.1006/jmbi.1996.0570 (1996).
53 Kaizuka, Y., Douglass, A. D., Vardhana, S., Dustin, M. L. & Vale, R. D. The coreceptor CD2 uses plasma membrane microdomains to transduce signals in T cells. J Cell Biol 185, 521-534, doi:10.1083/jcb.200809136 (2009).
54 Zhao, Z. et al. Structural Design of Engineered Costimulation Determines Tumor Rejection Kinetics and Persistence of CAR T Cells. Cancer Cell 28, 415-428, doi:10.1016/j.ccell.2015.09.004 (2015).
55 Schneider, M. et al. Alterations of the CD58 gene in classical Hodgkin lymphoma. Genes Chromosomes Cancer 54, 638-645, doi:10.1002/gcc.22276 (2015).
56 Abdul Razak, F. R., Diepstra, A., Visser, L. & van den Berg, A. CD58 mutations are common in Hodgkin lymphoma cell lines and loss of CD58 expression in tumor cells occurs in Hodgkin lymphoma patients who relapse. Genes Immun 17, 363-366, doi:10.1038/gene.2016.30 (2016).
57 Sembries, S., Pahl, H., Stilgenbauer, S., Döhner, H. & Schriever, F. Reduced Expression of Adhesion Molecules and Cell Signaling Receptors by Chronic Lymphocytic Leukemia Cells With 11q Deletion. Blood 93, 624-631, doi:10.1182/blood.V93.2.624 (1999).
58 Barker, H. F., Hamilton, M. S., Ball, J., Drew, M. & Franklin, I. M. Expression of adhesion molecules LFA-3 and N-CAM on normal and malignant human plasma cells. Br J Haematol 81, 331-335, doi:10.1111/j.1365-2141.1992.tb08236.x (1992).
59 Koretz, K., Schlag, P. & Moller, P. Sporadic loss of leucocyte-function-associated antigen-3 (LFA-3) in colorectal carcinomas. Virchows Arch A Pathol Anat Histopathol 419, 389-394, doi:10.1007/BF01605072 (1991).
60 Siliciano, R. F., Pratt, J. C., Schmidt, R. E., Ritz, J. & Reinherz, E. L. Activation of cytolytic T lymphocyte and natural killer cell function through the T11 sheep erythrocyte binding protein. Nature 317, 428-430, doi:10.1038/317428a0 (1985).
61 Liu, L. L. et al. Critical Role of CD2 Co-stimulation in Adaptive Natural Killer Cell Responses Revealed in NKG2C-Deficient Humans. Cell Rep 15, 1088-1099, doi:10.1016/j.celrep.2016.04.005 (2016).
62 Liu, E. et al. Use of CAR-Transduced Natural Killer Cells in CD19-Positive Lymphoid Tumors. N Engl J Med 382, 545-553, doi:10.1056/NEJMoa1910607 (2020).

1.-63. (canceled)

64. A method for (i) determining the responsiveness of an individual to a CAR-T cell therapy, (ii) identifying an individual who has an increased unresponsiveness to a CAR-T cell therapy, (iii) optimizing the therapeutic efficacy of a CAR-T cell therapy in an individual, (iv) administering a CAR-T cell therapy to an individual, and/or (v) treating a health condition in an individual, the method comprising:
a) detecting whether an expression level of CD58 is decreased compared to a reference expression level of CD58 or lost or one or more molecular alterations in CD58 activity is present in a biological sample obtained from the individual, wherein said detecting comprises contacting the biological sample with a detection reagent and detecting an interaction between the detection reagent with a CD58 encoding gene or a product thereof in the sample; and
b) (i) identifying the individual as having decreased responsiveness to treatment with the CAR-T cell therapy if the expression level of CD58 is decreased compared to a reference expression level of CD58 or lost or at least one of the one or more molecular alterations in CD58 activity is detected in the sample,
(ii) selecting the individual as having increased unresponsiveness to treatment with a CAR-T cell therapy if the expression level of CD58 is decreased compared to a reference expression level of CD58 or lost or at least one of the one or more molecular alterations in CD58 activity is detected in the sample; or
(iii) identifying a therapeutically effective amount of the CAR-T cell therapy based on the detected interaction between the detection reagent with the CD58 encoding gene or a product thereof; or
(iv) administering a therapeutically effective amount of the CAR-T cell therapy based on the detected interaction between the detection reagent with the CD58 encoding gene or a product thereof; or
(v) administering a treatment with a CAR construct comprising a CD2 signaling domain to the individual based on the detection of a decreased or lost expression level of CD58 or one or more molecular alterations in the CD58-encoding gene in step (a).

65. The method of claim 64, the individual has or is suspected of having a health condition associated with (i) a decreased level or loss of CD58 expression compared to a reference expression level of CD58 or (ii) with one or more molecular alterations in CD58 activity.

66. The method of claim 65, further comprising treating the health condition.

67. The method of claim 64, wherein the health condition is a proliferative disorder selected from the group consisting of a solid tumor cancer, a non-solid tumor cancer, and a hematological malignancy.

68. The method of claim 64, wherein the one or more molecular alterations in CD58 activity is selected from the group consisting of an increased level of RNA/protein expression, a reduced level of RNA/protein expression, a loss of expression, an aberrant RNA/protein expression profile, a single nucleotide polymorphism (SNP), a single-nucleotide variant (SNV), a gene amplification, a gene rearrangement, a gene fusion, a deletion, a frameshift deletion, an insertion, an InDel mutation, an epigenetic alteration, an amino acid substitution, and combinations of any thereof.

69. The method of claim 64, wherein the one or more molecular alterations comprises a loss of CD58 expression, a reduced expression of CD58 compared to a reference expression level of CD58, or expression of a mutated form of CD58.

70. The method of claim 64, wherein the one or more molecular alterations comprises an amino acid substitution at a position corresponding to K60 of SEQ ID NO: 1.

71. The method of claim 70, wherein the amino acid substitution is a Lys-to-Glu substitution (K60E).

72. The method of claim 64, wherein the one or more molecular alterations comprises an amino acid substitution at a position corresponding to C187 of SEQ ID NO: 1.

73. The method of claim 72, wherein the amino acid substitution is a Cys-to-Arg substitution (C187R).

74. The method of claim 64, wherein the one or more molecular alterations in the CD58-encoding gene or a product thereof comprises a reduced binding affinity of a CD58 protein product to its ligand CD2.

75. The method of claim 64, further comprising administering the CAR-T cell therapy to the individual, wherein the CAR-T cell therapy is administered to the individual as single therapy or in combination with one or more additional therapies.

76. The method of claim 75, wherein the CAR-T cell therapy targets an antigen expressed by a cancer cell.

77. The method of claim 76, wherein the cancer is a B-cell malignancy.

78. The method of claim 77, wherein the CAR-T cell therapy targeting the antigen expressed by the B-cell malignancy comprises a CAR construct comprising a CD2 signaling domain.

79. The method of claim 78, wherein the antigen expressed by the B-cell malignancy is CD19.

80. The method of claim 79, wherein the CAR construct comprising a CD2 signaling domain further comprises an anti-CD19 scFv domain.

81. The method of claim 64, wherein the CAR-T cell therapy targets an antigen that is expressed at low density compared to its expression density in a wild-type cell.

82. The method of claim 78, wherein the CAR construct comprising a CD2 signaling domain comprises the amino acid sequence of SEQ ID NO: 4.

83. The method of claim 82, wherein the one or more molecular alterations in the CD58-encoding gene or a product thereof comprises a reduced binding affinity of a CD58 protein product to its ligand CD2.

84. A kit for the diagnosis and/or treatment of a health condition in an individual, the kit comprising (i) reagents for assessing an expression level of CD58 or the presence and/or absence of one or more molecular alterations of a CD58-encoding gene or a product thereof in a biological sample from the individual, and (ii) instructions for use thereof,
wherein the kit is further configured for (i) determining the responsiveness of the individual to a CAR-T cell therapy (ii) identifying an individual who has an increased unresponsiveness to a CAR-T cell therapy, (iii) optimizing the therapeutic efficacy of a CAR-T cell therapy in an individual, (iv) administering a CAR-T cell therapy to an individual, and/or (v) treating a health condition in an individual.

85. A genetic-based system for diagnosis and/or treatment of health conditions comprising:
a) a logic processor;
b) a stored program code that is executable by the logic processor, which when executed by the processor provides operations for performing one or more of the following:
i) determining the responsiveness of an individual to a CAR-T cell therapy;
ii) identifying an individual as having increased unresponsiveness to treatment with a CAR-T cell therapy;
iii) optimizing the therapeutic efficacy of a CAR-T cell therapy in an individual; and
iv) calculating or administering a therapeutically effective amount of a CAR-T cell therapy to an individual.

86. A non-transitory computer readable medium containing machine executable instructions that when executed cause a processor to perform operations comprising:
receiving a profile comprising a preselected set of data input;
assigning, based at least in part on the profile, (i) a relative performance score to the identified CAR-T cell therapy, and/or (ii) a relative unresponsiveness score to the identified individual; and
outputting a report for the CAR-T cell therapy based upon the assigned performance score and/or for the individual based upon the assigned unresponsiveness score.
